(19) United States
(12) Patent Application Publication
(10) Pub. No.: US 2005/0287528 A1
(43) Pub. Date: Dec. 29, 2005

(54) Methods for detecting ovarian cancer

(75) Inventors: Eleftherios P Diamandis, Toronto, ON (CA); Tadaaki Kishi, Toronto,ON (CA)

Correspondence Address: MERCHANT & GOULD PC, P.O. BOX 2903, MINNEAPOLIS, MN, 55402-0903, US

(73) Assignee: Mount Sinai Hospital, Toronto, ON (CA)

(21) Appl. No.: 10/510,321

(22) PCT Filed: Apr. 4, 2003
(86) PCT No.: PCT/CA03/00495

§ 371 Date: May 25, 2005

Related U.S. Application Data
(60) Provisional application No. 60/370,559, filed on Apr. 4, 2002.

(51) Int. Cl. C12Q 1/68; G01N 33/574

(52) U.S. Cl. 435/6; 435/7.23

(57) ABSTRACT

A method for detecting a kallikrein 8 polypeptide associated with ovarian cancer in a patient is provided comprising detecting kallikrein 8 polypeptide in a sample from the patient. Methods are also provided for monitoring the progression of ovarian cancer in a patient, determining whether ovarian cancer has metastasized or is likely to metastasize in the future, and assessing the aggressiveness or indolence of ovarian cancer. In addition, methods for assessing the potential efficacy of a test agent for inhibiting ovarian cancer in a patient, selecting an agent for inhibiting ovarian cancer in a patient, and assessing the ovarian cancer cell carcinogenic potential of a test compound are described. The invention also contemplates a method of treating a patient susceptible to, or having a cancer that expresses a kallikrein 8 polypeptide.

### FIELD OF THE INVENTION

The invention relates to methods and compositions for detecting ovarian cancer.

### BACKGROUND OF THE INVENTION

Ovarian cancer represents a great clinical challenge in gynecological oncology. Since most patients are asymptomatic until the disease has metastasized, two-thirds are diagnosed with advanced disease (1). In the United States, around 23,000 new cases of ovarian cancer and about 14,000 deaths from the disease were expected for the year 2000 (2), giving it the highest mortality rate of all gynecological malignancies.

The human kallikrein gene family, a subfamily of serine proteases, is now known to include fifteen members (1;2). All genes in this family localize to chromosome 19q13.4 and share significant similarities at both the DNA and amino acid level. The human kallikrein family includes hK3/prostate specific antigen (PSA) which is the most important biomarker for prostate cancer (3). Recently, three other members of this family, hK6/neurosin, hK10/normal epithelial cell specific 1 (NES1) and hK11/trypsin like serine protease (TLSP), have been shown to be potential biomarkers for ovarian and prostate cancer (4-6). In addition, recent reports also suggest that many other members of this family are associated with cancers of the breast, ovary, prostate and testis, as well as with diverse diseases of the central nervous system, skin, etc. (reviewed in ref. (7)).

KLK8/neuropsin is a member of human kallikrein family (1;2) (note: KLK8, gene; hK8, protein, according to the official kallikrein gene nomenclature (8)). Originally, KLK8 was cloned from a human skin cDNA library as a homolog of mouse neuropsin (9). The mouse homrolog has highest expression in skin and brain, especially the hippocampus, and was assumed to be associated with neural plasticity, memory formation and some forms of epilepsy (10-13). KLK8 mRNA is increased in Alzheimer's disease hippocampus compared to controls, which suggests that KLK8 may indeed have a relationship with neural plasticity in humans (14). KLK8 transcripts in ovarian cancer tissues are expressed at higher levels than in controls (15). Two splice variants of KLK8 have been detected in ovarian cancer (16).

To date, there is no literature describing any relationship between hK8 protein expression and cancer.

### SUMMARY OF THE INVENTION

Recombinant human kallikrein 8 was produced using a baculovirus expression system, purified using column chromatography, and injected into mice and rabbits for antibody generation. These antibodies were used to develop a highly sensitive and specific immunoassay for hK8. The assay was applied to the measurement of native hK8 in tissue extracts and biological fluids. Applicants found elevated levels of serum hK8 in patients with ovarian cancer. In addition, hK8 was found in tumor extracts and ascites fluid of patients with ovarian cancer.

Therefore, kallikrein 8 has particular application in the detection of ovarian cancer. Thus, kallikrein 8 constitutes a new biomarker for diagnosis and monitoring (i.e. monitoring progression or therapeutic treatment) of ovarian cancer. In accordance with an aspect of the invention kallikrein 8 is used for the diagnosis, monitoring, and prognosis of ovarian cancer, and it may be used as a biomarker before surgery or after relapse.

The presence of kallikrein 8 may be assessed, for example by detecting the presence in the sample of (a) polypeptides or polypeptide fragments corresponding to the marker; and/or (b) metabolites which are produced directly or indirectly by polypeptides corresponding to the marker.

In an aspect of the invention kallikrein 8 and agents that bind to kallikrein 8 may be used to detect ovarian cancer and they can be used in the diagnostic evaluation of ovarian cancer, and the identification of subjects with a predisposition to such disorders. The present invention therefore relates to a method for diagnosing and monitoring ovarian cancer in a subject comprising detecting kallikrein 8 in a sample from the subject

The term “detect” or “detecting” includes assaying, identifying, imaging or otherwise establishing the presence or absence of the target kallikrein 8, subunits thereof, or combinations of reagent bound targets, and the like, or assaying for, imaging, ascertaining, establishing, or otherwise determining one or more factual characteristics of ovarian cancer, metastasis, stage, or similar conditions. The term encompasses diagnostic, prognostic, and monitoring applications for kallikrein 8.

In an aspect of the invention, a method for screening a subject for ovarian cancer is provided comprising (a) obtaining a biological sample from a subject; (b) detecting the amount of kallikrein 8 in said sample; and (c) comparing said amount of kallikrein 8 detected to a predetermined standard, where detection of a level of kallikrein 8 greater than that of a standard indicates disease.

In an embodiment, the invention provides a method for detecting a kallikrein 8 polypeptide associated with ovarian cancer in a patient comprising:
(a) obtaining a sample from a patient;
(b) detecting or identifying in the sample a kallikrein 8 polypeptide associated with ovarian cancer; and
(c) comparing the detected amounts with amounts detected for a standard.

In an aspect the invention provides a method of assessing whether a patient is afflicted with ovarian cancer (e.g. screening, detection of a recurrence, reflex testing), the method comprises comparing:
(a) levels of a kallikrein 8 polypeptide associated with ovarian cancer in a sample from the patient; and
(b) normal levels of the kallikrein 8 polypeptide in a control non-ovarian cancer sample.

A significant difference between the levels of the kallikrein 8 polypeptide in the patient sample and the normal levels is an indication that the patient is afflicted with ovarian cancer.

In another aspect, the invention provides a method of assessing whether a patient is afflicted with or has a pre-disposition for ovarian cancer, the method comprising comparing:
(a) levels of a kallikrein 8 polypeptide associated with ovarian cancer in a sample from the patient; and
(b) normal levels of the kallikrein 8 polypeptide in samples of the same type obtained from control patients not afflicted with ovarian cancer, wherein significantly altered levels of the kallikrein 8 polypeptide, relative to the corresponding normal levels of the kallikrein 8 polypeptide, is an indication that the patient is afflicted with ovarian cancer.

Kallikrein 8 may be measured using an agent or reagent that detects or binds to kallikrein 8, preferably antibodies specifically reactive with kallikrein 8 or a part thereof.

In an aspect, the invention relates to a method for diagnosing and monitoring ovarian cancer in a subject by quantitating kallikrein 8 in a biological sample from the subject comprising (a) reacting the biological sample with a binding agent specific for kallikrein 8 (e.g. an antibody) which is directly or indirectly labelled with a detectable substance; and (b) detecting the detectable substance.

Embodiments of the methods of the invention may comprise (a) reacting a biological sample from a subject with an antibody specific for kallikrein 8 which is directly or indirectly labelled with an enzyme; (b) adding a substrate for the enzyme wherein the substrate is selected so that the substrate, or a reaction product of the enzyme and substrate forms fluorescent complexes; (c) quantitating kallikrein 8 in the sample by measuring fluorescence of the fluorescent complexes; and (d) comparing the quantitated levels to levels obtained for other samples from the subject patient, or control subjects. In an embodiment, the quantitated levels are compared to levels quantitated for control subjects without ovarian cancer wherein an increase in kallikrein 8 levels compared with the control subjects is indicative of disease.

In a particular embodiment of the invention, a method for detecting ovarian cancer comprises the following steps
(a) incubating a biological sample with a first antibody specific for kallikrein 8 which is directly or indirectly labeled with a detectable substance, and a second antibody specific for kallikrein 8 which is immobilized;
(b) separating the first antibody from the second antibody to provide a first antibody phase and a second antibody phase;
(c) detecting the detectable substance in the first or second antibody phase thereby quantitating kallikrein 8 in the biological sample; and
(d) comparing the quantitated kallikrein 8 with levels for a predetermined standard.

The standard may correspond to levels quantitated for samples from control subjects without ovarian cancer, with a different disease stage, or from other samples of the subject. In accordance with an aspect of the invention, increased levels of kallikrein 8 as compared to a standard is indicative of ovarian cancer.

The invention also contemplates the methods described herein using multiple markers for ovarian cancer. Therefore, the invention contemplates a method for anaylzing a biological sample for the presence of kallikrein 8 and other markers that are specific indicators of ovarian cancer. Other markers include markers to kallikreins such as human stratum corneum chymotryptic enzyme (HSCCE), kallikrein 2, kallikrein 3, kallikrein 4, kallikrein 5, kallikrein 6, kallikrein 9, kallikrein 10, and kallikrein 11; CA125, CA15-3, CA72-4, CA19-9, OVX1, lysophosphatidic acid (LPA), creatin-kinase BB, haptoglobin alpha, prostasin, osteopontin, and carcinoembryonic antigen (CEA). The methods described herein may be modified by including reagents to detect the markers.

The invention further relates to a method of assessing the efficacy of a therapy for inhibiting ovarian cancer in a patient This method comprises comparing:
(a) levels of a kallikrein 8 polypeptide associated with ovarian cancer in a sample from the patient; and
(b) levels of the kallikrein 8 polypeptide in a second sample obtained from the patient following therapy.

A significant difference between the levels of the kallikrein 8 polypeptide in the second sample, relative to the first sample, is an indication that the therapy is efficacious for inhibiting ovarian cancer.

The “therapy” may be any therapy for treating ovarian cancer including but not limited to chemotherapy, immunotherapy, gene therapy, radiation therapy, and surgical removal of tissue. Therefore, the method can be used to evaluate a patient before, during, and after therapy, for example, to evaluate the reduction in tumor burden.

In an aspect, the invention provides a method for monitoring the progression of ovarian cancer in a patient, the method comprising:
(a) detecting in a patient sample at a first time point, a kallikrein 8 polypeptide associated with ovarian cancer in a sample from the patient; and
(b) repeating step (a) at a subsequent point in time; and
(c) comparing the levels detected in (a) and (b), and therefrom monitoring the progression of ovarian cancer in the patient.

In another aspect, the invention provides a method for assessing the aggressiveness or indolence of ovarian cancer (e.g. staging), the method comprising comparing:
(a) levels of a kallikrein 8 polypeptide associated with ovarian cancer in a sample from the patient; and
(b) normal levels of the kallikrein 8 polypeptide in a control sample.

A significant difference between the levels in the sample and the normal levels is an indication that the cancer is aggressive or indolent.

The invention provides a method for determining whether an ovarian cancer has metastasized or is likely to metastasize in the future, the method comprising comparing:
(a) levels of a kallikrein 8 polypeptide associated with ovarian cancer in a sample from the patient; and
(b) normal levels (or non-metastatic levels) of the kallikrein 8 polypeptide in a control sample.

A significant difference between the levels in the patient sample and the normal levels is an indication that the cancer has metastasized or is likely to metastasize in the future.

The invention also provides a method for assessing the potential efficacy of a test agent for inhibiting ovarian cancer in a patient, and a method of selecting an agent for inhibiting ovarian cancer in a patient.

The invention further provides a method of inhibiting ovarian cancer in a patient comprising:
(a) obtaining a sample comprising cancer cells from the patient;
(b) separately maintaining aliquots of the sample in the presence of a plurality of test agents;
(c) comparing levels of a kallikrein 8 polypeptide associated with ovarian cancer in each of the aliquots; and
(d) administering to the patient at least one of the test agents which alters the levels of the kallikrein 8 polypeptide in the aliquot containing that test agent, relative to other test agents.

The invention also contemplates a method of assessing the ovarian cancer carcinogenic potential of a test compound comprising:
(a) maintaining separate aliquots of ovarian cancer cells in the presence and absence of the test compound; and
(b) comparing levels of a kallikrein 8 polypeptide in each of the aliquots.

A significant difference between the levels of the kallikrein 8 polypeptide in the aliquot maintained in the presence of (or exposed to) the test compound relative to the aliquot maintained in the absence of the test compound, indicates that the test compound possesses ovarian cancer carcinogenic potential.

The invention also provides a diagnostic composition comprising a kallikrein 8 polypeptide or agents that bind to the polypeptide. In an embodiment, the composition comprises an agent that binds a kallikrein 8 polypeptide or a fragment thereof. An agent may be labeled with a detectable substance.

Still further the invention provides therapeutic applications for ovarian cancer employing a kallikrein 8 polypeptide and/or binding agents for the polypeptide.

In accordance with an aspect of the invention an in vivo method is provided comprising administering to a subject an agent that has been constructed to target one or more kallikreins.

The invention therefore contemplates an in vivo method comprising administering to a mammal one or more agent that carries a label for imaging and binds to a kallikrein, preferably kallikrein 8, and then imaging the mammal.

According to a preferred aspect of the invention, an in vivo method for imaging ovarian cancer is provided comprising:
(a) injecting a patient with an agent that binds to kallikrein 8, the agent carrying a label for imaging the ovarian cancer;
(b) allowing the agent to incubate in vivo and bind to kallikrein 8 associated with the ovarian cancer; and
(c) detecting the presence of the label localized to the ovarian cancer.

In an embodiment of the invention the agent is an antibody which recognizes the kallikrein. In another embodiment of the invention the agent is a chemical entity which recognizes the kallikrein.

The agent carries a label to image the kallikreins. Examples of labels useful for imaging are radiolabels, fluorescent labels (e.g fluorescein and rhodamine), nuclear magnetic resonance active labels, positron emitting isotopes detectable by a positron emission tomography (“PET”) scanner, chemiluminescers such as luciferin, and enzymatic markers such as peroxidase or phosphatase. Short-range radiation emitters, such as isotopes detectable by short-range detector probes can also be employed.

The invention also contemplates the localization or imaging methods described herein using multiple markers for ovarian cancer. For example, a method for imaging ovarian cancer may further comprise injecting the patient with one or more of an agent that binds to human stratum corneum chymotryptic enzyme (HSCCE), kallikrein 2, kallikrein 3, kallikrein 4, kallikrein 5, kallikrein 6, kallikrein 9, kallikrein 10, kallikrein 11, CA125, CA15-3, CA19-9, CA72-4, OVX1, creatin-kinase BB, haptoglobin alpha, lysophosphatidic acid (LPA), osteopontin, prostasin, or carcinoembryonic antigen (CEA), preferably CA125.

The invention also relates to kits for carrying out the methods of the invention. In an embodiment, the kit is for assessing whether a patient is afflicted with ovarian cancer and it comprises reagents for assessing kallikrein 8 polypeptides.

In another aspect the invention relates to a kit for assessing the suitability of each of a plurality of test compounds for inhibiting ovarian cancer in a patient. The kit comprises reagents for assessing kallikrein 8 polypeptides. The kit may also comprise a plurality of test agents or compounds.

The invention contemplates a kit for assessing the presence of ovarian cancer cells, wherein the kit comprises antibodies specific for a kallikrein 8 polypeptide.

Additionally the invention provides a kit for assessing the ovarian cancer carcinogenic potential of a test compound. The kit comprises ovarian cancer cells and reagents for assessing kallikrein 8.

Other objects, features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### DESCRIPTION OF THE DRAWINGS

The invention will now be described in relation to the drawings in which:

FIG. 1: SDS-PAGE of purified recombinant hK8 at each chromatographic step. Each lane (except marker) was loaded with 3 μg of total protein hK8 is essentially pure after the benzamidine step (see also Table 1).

FIG. 2: A typical calibration curve for the hK8 immunoassay. The background fluorescence (zero calibrator) was subtracted from all measurements. The dynamic range of this assay is 0.2-20 μg/L.

FIG. 3: Specificity of the hK8 immunoassay. The assay was performed in the presence of both mouse and rabbit antibodies, or in the absence of mouse, or rabbit antibody (substituted by non-immune serum). Samples tested were amniotic fluid and tissue extracts from esophagus and tonsil.

FIG. 4: Hormonal regulation of hK8 in the prostate carcinoma cell line PC-3 (AR)₆ and the breast carcinoma cell line MCF-7. In the PC-3 (AR)₆ cell line, hK8 is up-regulated by norgestrel (an androgenic progestin) and dihydrotestosterone (DHT). In the MCF-7 cell line, hK8 is up-regulated by estradiol.

FIG. 5: Expression of hK8 in adult and fetal tissue extracts from males and females.

FIG. 6: Levels of hK8 (μ/g of total protein) in ascites fluid of women with advanced ovarian cancer. Higher levels are seen in lower grade disease. The p value was calculated by the Kruskal Wallis test. Horizontal lines indicate median values.

FIG. 7: Kaplan-Meier survival curves of patients with advanced ovarian carcinoma. Patients were categorized as hK8-negative (ascites fluid hK8 concentration<25^{th} percentile; 0.75 μg/g of total protein) or hK8-positive (hk8 concentration>0.75 μg/g). The p value was calculated by the log-rank test

FIG. 8: hK8 serum concentration in 26 ovarian cancer patients and 25 normal females. At 95% specificity (cutoff of 5.5 μg/L, indicated by the dotted lines), the sensitivity is 54%.

FIG. 9: Monitoring of an ovarian cancer patient with serum CA 125 and hK8.

FIG. 10: High-performance liquid chromatographic separation on a gel filtration column of a serum from an ovarian cancer patient (A), an esophageal extract (B), an aminiotic fluid (C) and a breast milk (D). The peak represents the free 30 kDa form of hK8.

### DETAILED DESCRIPTION OF THE INVENTION

The invention relates to newly discovered correlations between expression of kallikrein 8 and ovarian cancer. The kallikrein 8 marker provides sensitive methods for detecting ovarian cancer. The levels of expression of kallikrein 8 correlate with the presence of ovarian cancer or a pre-malignant condition in a patient. Methods are provided for detecting the presence of ovarian cancer in a sample, the absence of ovarian cancer in a sample, the stage of an ovarian cancer, the grade of an ovarian cancer, the benign or malignant nature of an ovarian cancer, the metastatic potential of an ovarian cancer, assessing the histological type of neoplasm associated with the ovarian cancer, the indolence or aggressiveness of the cancer, and other characteristics of ovarian cancer that are relevant to prevention, diagnosis, characterization, and therapy of ovarian cancer in a patient. Methods are also provided for assessing the efficacy of one or more test agents for inhibiting ovarian cancer, assessing the efficacy of a therapy for ovarian cancer, monitoring the progression of ovarian cancer, selecting an agent or therapy for inhibiting ovarian cancer, treating a patient afflicted with ovarian cancer, inhibiting ovarian cancer in a patient, and assessing the carcinogenic potential of a test compound.

In accordance with the present invention there may be employed conventional molecular biology, microbiology, and recombinant DNA techniques within the skill of the art. Such techniques are explained fully in the literature. See for example, Sambrook, Fritsch, & Maniatis (Molecular Cloning: A Laboratory Manual, Second Edition (1989) Cold Spring Harbor Laboratory Press, Cold Spring Harbor, N.Y); DNA Cloning: A Practical Approach, Volumes I and II (D. N. Glover ed. 1985); Oligonucleotide Synthesis (M. J. Gait ed. 1984); Nucleic Acid Hybridization B. D. Hames & S. J. Higgins eds. (1985); Transcription and Translation B. D. Hames & S. J. Higgins eds. (1984); Animal Cell Culture R. I. Freshney, ed. (1986); Immobilized Cells and enzymes IRL Press, (1986); and B. Perbal, A Practical Guide to Molecular Cloning (1984). The invention may also employ standard methods in immunology known in the art such as described in Stites et al. (eds) Basic and Clinical Immunology, 8^{th} Ed., Appleton & Lange, Norwalk, Conn. (1994) and Mishell and Shigi (eds), Selected Methods in Cellular Immunology, W.H. Freeman and Co., New York (1980).

Glossary

For convenience, certain terms employed in the specification and claims are collected here.

The terms “sample”, “biological sample”, and the like mean a material known or suspected of expressing or containing a kallikrein 8 polypeptide associated with ovarian cancer. The test sample can be used directly as obtained from the source or following a pretreatment to modify the character of the sample. The sample can be derived from any biological source, such as tissues, extracts, or cell cultures, including cells (e.g. tumor cells), cell lysates, and physiological fluids, such as, for example, whole blood, plasma, serum, saliva, ocular lens fluid, cerebral spinal fluid, sweat, urine, milk, ascites fluid, synovial fluid, peritoneal fluid and the like. The sample can be obtained from animals, preferably mammals, most preferably humans. The sample can be treated prior to use, such as preparing plasma from blood, diluting viscous fluids, and the like. Methods of treatment can involve filtration, distillation, extraction, concentration, inactivation of interfering components, the addition of reagents, and the like. Proteins may be isolated from the samples and utilized in the methods of the invention. In an embodiment, the sample is a serum sample.

“Kallikrein 8 polypeptide(s)”, “kallikrein 8 marker(s)”, or “kallikrein 8” includes native-sequence polypeptides, isoforms, precursors, proproteins, and chimeric polypeptides.

A “native-sequence polypeptide” comprises a polypeptide having the same amino acid sequence of a polypeptide derived from nature. Such native-sequence polypeptides can be isolated from nature or can be produced by recombinant or synthetic means. A native-sequence polypeptide may comprise a proprotein or precursor.

The amino acid sequences for a native kallikrein 8 polypeptide employed or detected in accordance with the present invention include the sequences found in Yoshida et al, and GenBank for human kallikrein 8 (“hK8”) at GenBank Accession Nos. NP_{—}009127, BAA28676, BAA28673, NP_{—}653088, NP 653089, NP 653090, O60259, and AAG23254 (see for example SEQ ID NOs.1 and 2) or a portion thereof. Other useful kallikrein 8 polypeptides are substantially identical to these sequences (e.g. at least about 45%, preferably 50%, 55%, 60%, 65%, 70%, 75%, 80%, 85%, 90%, and 95%, more preferably at least 97%, 98%, or 99% sequence identity), and preferably retain the immunogenic activity of the corresponding native-sequence kallikrein 8 polypeptide.

The term “native-sequence polypeptide” also specifically encompasses naturally occurring truncated or secreted forms of a kallikrein 8 polypeptide, polypeptide variants including naturally occurring variant forms (e.g., alternatively spliced forms or splice variants), and naturally occurring allelic variants.

The term “polypeptide variant” means a polypeptide having at least about 70-80%, preferably at least about 85%, more preferably at least about 90%, most preferably at least about 95% amino acid sequence identity with a native-sequence polypeptide, in particular having at least 70-80%, 85%, 90%, 95% amino acid sequence identity to a sequence identified in Yoshida et al, and GenBank Accession Nos. NP_{—}009127, BAA28676, BAA28673, NP-653088, NP 653089, NP_{—}653090, O60259, and AAG23254 (see for example SEQ ID NOs.1 and 2). Such variants include, for instance, polypeptides wherein one or more amino acid residues are added to, or deleted from, the N- or C-terminus of the full-length or mature sequences of BAA28676, BAA28673, and AAG23254 (e.g. SEQ ID NOs.1 and 2), including variants from other species, but excludes a native-sequence polypeptide.

An allelic variant may also be created by introducing substitutions, additions, or deletions into a nucleic acid encoding a native polypeptide sequence such that one or more amino acid substitutions, additions, or deletions are introduced into the encoded protein. Mutations may be introduced by standard methods, such as site-directed mutagenesis and PCR-mediated mutagenesis. In an embodiment, conservative substitutions are made at one or more predicted non-essential amino acid residues. A “conservative amino acid substitution” is one in which an animo acid residue is replaced with an amino acid residue with a similar side chain. Amino acids with similar side chains are known in the art and include amino acids with basic side chains (e.g. Lys, Arg, His), acidic side chains (e.g. Asp, Glu), uncharged polar side chains (e.g. Gly, Asp, Glu, Ser, Thr, Tyr and Cys), nonpolar side chains (e.g. Ala, Val, Leu, Iso, Pro, Trp), beta-branched side chains (e.g. Thr, Val, Iso), and aromatic side chains (e.g. Tyr, Phe, Trp, His). Mutations can also be introduced randomly along part or all of the native sequence, for example, by saturation mutagenesis. Following mutagenesis the variant polypeptide can be recombinantly expressed and the activity of the polypeptide may be determined.

Polypeptide variants include polypeptides comprising amino acid sequences sufficiently identical to or derived from the amino acid sequence of a native polypeptide which include fewer amino acids than the full length polypeptides. A portion of a polypeptide can be a polypeptide which is for example, 10, 15, 20, 25, 30, 35, 40, 45, 50, 60, 70, 80, 90, 100 or more amino acids in length. Portions in which regions of a polypeptide are deleted can be prepared by recombinant techniques and can be evaluated for one or more functional activities such as the ability to form antibodies specific for a polypeptide.

A naturally occurring allelic variant may contain conservative amino acid substitutions from the native polypeptide sequence or it may contain a substitution of an amino acid from a corresponding position in a kallikrein polypeptide homolog, for example, the murine kallikrein polypeptide.

Percent identity of two amino acid sequences, or of two nucleic acid sequences identified herein is defined as the percentage of amino acid residues or nucleotides in a candidate sequence that are identical with the amino acid residues in a kallikrein polypeptide or nucleic acid sequence, after aligning the sequences and introducing gaps, if necessary, to achieve the maximum percent sequence identity, and not considering any conservative substitutions as part of the sequence identity. Alignment for purposes of determining percent amino acid or nucleic acid sequence identity can be achieved in various conventional ways, for instance, using publicly available computer software including the GCG program package (Devereux J. et al., Nucleic Acids Research 12(1): 387, 1984); BLASTP, BLASTN, and FASTA (Atschul, S. F. et al. J. Molec. Biol. 215: 403-410, 1990). The BLAST X program is publicly available from NCBI and other sources (BLAST Manual, Altschul, S. et al. NCBI NLM NIH Bethesda, Md. 20894; Altschul, S. et al. J. Mol. Biol. 215: 403-410, 1990). Skilled artisans can determine appropriate parameters for measuring alignment, including any algorithms needed to achieve maximal alignment over the full length of the sequences being compared. Methods to determine identity and similarity are codified in publicly available computer programs.

Kallikrein 8 polypeptides include chimeric or fusion proteins. A “chimeric protein” or “fusion protein” comprises all or part (preferably biologically active) of a kallikrein 8 polypeptide operably linked to a heterologous polypeptide (i.e., a polypeptide other than a kallikrein 8 polypeptide). Within the fusion protein, the term “operably linked” is intended to indicate that the kallikrein polypeptide and the heterologous polypeptide are fused in-frame to each other. The heterologous polypeptide can be fused to the N-terminus or C-terminus of the kallikrein polypeptide. A useful fusion protein is a GST fusion protein in which a kallikrein polypeptide is fused to the C-terminus of GST sequences. Another example of a fusion protein is an immunoglobulin fusion protein in which all or part of a kallikrein polypeptide is fused to sequences derived from a member of the immunoglobulin protein family. Chimeric and fusion proteins can be produced by standard recombinant DNA techniques.

A kallikrein 8 polypeptide may be part of a complex, in particular, a complex with a protease inhibitor.

Kallikrein 8 polypeptides may be isolated from a variety of sources, such as from human tissue types or from another source, or prepared by recombinant or synthetic methods, or by any combination of these and similar techniques.

The term “subject” or “patient” refers to a warm-blooded animal such as a mammal which is afflicted with ovarian cancer or condition as described herein. Preferably, “subject” refers to a human.

“Kallikrein 8 polynucleotide(s)”, “kallikrein 8 nucleic acid(s)”, “KLK8”, or “KLK8 nucleic acid(s)” includes nucleic acids that encode a native-sequence kallikrein 8 polypeptide, a polypeptide variant including a portion of a kallikrein 8 polypeptide, an isoform, precursor, and chimeric polypeptide.

The nucleic acid sequences encoding native kallikrein 8 polypeptides employed in the present invention include the nucleic acid sequences of Yoshida et al and GenBank Accession Nos. NP_{—}009127, AB009849, AB012761, NM_{—}007196, NM_{—}144505, NM_{—}144506, NM_{—}144507, and AC011473 (for example, SEQ ID NOs. 3 and 4), or a fragment thereof.

Polynucleotides encoding kallikrien 8 polypeptides include nucleic acid sequences complementary to these nucleic acids, and nucleic acids that are substantially identical to these sequences (e.g. at least about 45%, preferably 50%, 55%, 60%, 65%, 70%, 75%, 80%, 85%, 90%, 95%, 97%, 98%, or 99% sequence identity).

Kallikrein polynucleotides also include sequences which differ from a nucleic acid sequence of Yoshida et al, and GenBank Accession Nos. NP_{—}009127, AB009849, AB012761, NM_{—}007196, NM_{—}144505, NM_{—}144506, NM_{—}144507, and AC011473 (for example, SEQ ID NOs. 3 and 4), due to degeneracy in the genetic code. As one example, DNA sequence polymorphisms within the nucleotide sequence of a kallikrein 8 polypeptide may result in silent mutations that do not affect the amino acid sequence. Variations in one or more nucleotides may exist among individuals within a population due to natural allelic variation. DNA sequence polymorphisms may also occur which lead to changes in the amino acid sequence of a kallikrein 8 polypeptide.

Kallikrein 8 polynucleotides also include nucleic acids that hybridize under stringent conditions, preferably high stringency conditions to a nucleic acid sequence of Yoshida et al, and GenBank Accession Nos. NP 009127, AB009849, AB012761, NM-007196, NM_{—}144505, NM_{—}144506, NM_{—}144507, and AC011473 (for example, SEQ ID NOs. 3 and 4). Appropriate stringency conditions which promote DNA hybridization are known to those skilled in the art, or can be found in Current Protocols in Molecular Biology, John Wiley & Sons, N.Y. (1989), 6.3.1-6.3.6. For example, 6.0× sodium chloride/sodium citrate (SSC) at about 45° C., followed by a wash of 2.0×SSC at 50° C. may be employed. The stringency may be selected based on the conditions used in the wash step. By way of example, the salt concentration in the wash step can be selected from a high stringency of about 0.2×SSC at 50° C. In addition, the temperature in the wash step can be at high stringency conditions, at about 65° C.

Kallikrein polynucleotides also include truncated nucleic acids or nucleic acid fragments and variant forms of the nucleic acids that arise by alternative splicing of an mRNA corresponding to a DNA.

Kallikrien polynucleotides are intended to include DNA and RNA (e.g. mRNA) and can be either double stranded or single stranded. A polynucleotide may, but need not, include additional coding or non-coding sequences, or it may, but need not, be linked to other molecules and/or carrier or support materials. The polynucleotides may be of any length suitable for a particular method.

Methods

A variety of methods can be employed for the diagnostic and prognostic evaluation of ovarian cancer involving a kallikrein 8 polypeptide, and the identification of subjects with a predisposition to such disorders. Such methods may, for example, utilize binding agents (e.g. antibodies) directed against a kallikrein 8 polypeptide, including peptide fragments. In particular, the antibodies may be used, for example, for the detection of either an over- or an under-abundance of kallikrein 8 polypeptide relative to a non-disorder state or the presence of a modified (e.g., less than full length) kallikrein 8 polypeptide which correlates with a disorder state, or a progression toward a disorder state.

The invention also contemplates a method for detecting ovarian cancer comprising producing a profile of levels of kallikrein 8 polypeptides in cells from a patient and comparing the profile with a reference to identify a protein profile for the test cells indicative of disease.

The methods described herein may be used to evaluate the probability of the presence of malignant or pre-malignant cells, for example, in a group of cells freshly removed from a host. Such methods can be used to detect tumors, quantitate their growth, and help in the diagnosis and prognosis of disease. The methods can be used to detect the presence of cancer metastasis, as well as confirm the absence or removal of all tumor tissue following surgery, cancer chemotherapy, and/or radiation therapy. They can further be used to monitor cancer chemotherapy and tumor reappearance.

The methods described herein can be adapted for diagnosing and monitoring ovarian carcinoma by detecting a kallikrein 8 polypeptide in biological samples from a subject. These applications require that the amount of kallikrein 8 polypeptide quantitated in a sample from a subject being tested be compared to levels quantitated for another sample or an earlier sample from the subject, or levels quantitated for a control sample. Levels for control samples from healthy subjects or ovarian cancer subjects may be established by prospective and/or retrospective statistical studies. Healthy subjects who have no clinically evident disease or abnormalities may be selected for statistical studies. Diagnosis may be made by a finding of statistically different levels of kallikrein 8 polypeptide compared to a control sample or previous levels quantitated for the same subject.

“Statistically different levels” or “significant differences” in levels of a kallikrien 8 polypeptide in a patient sample compared to a control or standard (e.g. normal levels or levels in other samples from a patient) may represent levels that are higher or lower than the standard error of the detection assay, preferably the levels are at least about 1.5, 2, 3, 4, 5, or 6 times higher, respectively, than the control or standard.

Binding agents specific for a kallikrein 8 polypeptide may be used for a variety of diagnostic and assay applications. There are a variety of assay formats known to the skilled artisan for using a binding agent to detect a target molecule in a sample. (For example, see Harlow and Lane, Antibodies: A Laboratory Manual, Cold Spring Harbor Laboratory, 1988). In general, the presence or absence of an ovarian cancer in a subject may be determined by (a) contacting a sample from the subject with a binding agent for a kallikrein 8 polypeptide; (b) detecting in the sample levels of polypeptide that bind to the binding agent; and (c) comparing the levels of polypeptide with a predetermined standard or cut-off value.

“Binding agent” refers to a substance such as a polypeptide or antibody that specifically binds to a kallikrein 8 polypeptide. A substance “specifically binds” to a polypeptide if it reacts at a detectable level with the kallikrein 8 polypeptide, and does not react detectably with peptides containing unrelated sequences or sequences of different polypeptides. Binding properties may be assessed using an ELISA, which may be readily performed by those skilled in the art (see for example, Newton et al, Develop. Dynamics 197:1-13, 1993).

A binding agent may be a ribosome, with or without a peptide component, an RNA molecule, or a polypeptide. A binding agent may be a polypeptide that comprises a kallikrein 8 polypeptide sequence, a peptide variant thereof, or a non-peptide mimetic of such a sequence. By way of example a kallikrein 8 polypeptide sequence may be a peptide portion of a kallikrein 8 polypeptide that is capable of modulating a function mediated by the kallikrein 8 polypeptide.

In certain preferred embodiments, the binding agent is an antibody.

In an aspect the present invention provides a diagnostic method for monitoring or diagnosing ovarian cancer in a subject by quantitating a kallikrein 8 polypeptide in a biological sample from the subject comprising reacting the sample with antibodies specific for a kallikrein 8 polypeptide, which are directly or indirectly labelled with detectable substances, and detecting the detectable substances.

In an aspect of the invention, a method for detecting ovarian cancer is provided comprising:
(a) obtaining a sample suspected of containing a kallikrein 8 polypeptide associated with ovarian cancer;
(b) contacting said sample with antibodies that specifically bind a kallikrein 8 polypeptide under conditions effective to bind the antibodies and form complexes;
(c) measuring the amount of kallikrein 8 polypeptide present in the sample by quantitating the amount of the complexes; and
(d) comparing the amount of kallikrein 8 polypeptide present in the samples with the amount of polypeptide in a control, wherein a change or significant difference in the amount of polypeptide in the sample compared with the amount in the control is indicative of ovarian cancer.

In an embodiment, the invention contemplates a method for monitoring the progression of ovarian cancer in a subject, comprising:
(a) contacting antibodies which bind to a kallikrein 8 polypeptide, with a sample from the subject so as to form binary complexes comprising the antibodies and kallikrein 8 polypeptide in the sample;
(b) determining or detecting the presence or amount of complex formation in the sample;
(c) repeating steps (a) and (b) at a point later in time; and
(d) comparing the result of step (b) with the result of step (c), wherein a significant difference in the amount of complex formation is indicative of the stage and/or progression of the ovarian cancer in the subject.

The amount of complexes may also be compared to a value representative of the amount of the complexes from a subject not at risk of, or afflicted with, ovarian cancer at different stages.

Thus, antibodies specifically reactive with a kallikrein 8 polypeptide, or derivatives, such as enzyme conjugates or labeled derivatives, may be used to detect kallikrein 8 polypeptides in various samples (e.g. biological materials). They may be used as diagnostic or prognostic reagents and they may be used to detect abnormalities in the level of kallikrein 8 polypeptide expression, or abnormalities in the structure, and/or temporal, tissue, cellular, or subcellular location of kallikrein 8 polypeptide. Antibodies may also be used to screen potentially therapeutic compounds in vitro to determine their effects on disorders (e.g. ovarian cancer) involving a kallikrein 8 polypeptide, and other conditions. In vitro immunoassays may also be used to assess or monitor the efficacy of particular therapies.

Antibodies may be used in any known immunoassays which rely on the binding interaction between an antigenic determinant of a kallikrein 8 polypeptide and the antibodies. Examples of such assays are radioimmunoassays, enzyme immunoassays (e.g. ELISA), immunofluorescence, immunoprecipitation, latex agglutination, hemagglutination, and histochemical tests. The antibodies may be used to detect and quantify a kallikrein 8 polypeptide in a sample in order to diagnose and treat such pathological states. These terms are well understood by those skilled in the art. A person skilled in the art will know, or can readily discern, other immunoassay formats without undue experimentation.

In particular, the antibodies may be used in immunohistochemical analyses, for example, at the cellular and sub-subcellular level, to detect a kallikrein 8 polypeptide, to localize it to particular ovarian tumor cells and tissues, and to specific subcellular locations, and to quantitate the level of expression.

Antibodies for use in the present invention include monoclonal or polyclonal antibodies, immunologically active fragments (e.g. a Fab or (Fab)₂ fragments), antibody heavy chains, humanized antibodies, antibody light chains, genetically engineered single chain Fᵥ molecules (Ladner et al, U.S. Pat. No. 4,946,778), chimeric antibodies, for example, antibodies which contain the binding specificity of murine antibodies, but in which the remaining portions are of human origin, or derivatives, such as enzyme conjugates or labeled derivatives.

Antibodies including monoclonal and polyclonal antibodies, fragments and chimeras, may be prepared using methods known to those skilled in the art. An isolated native or recombinant kallikrein 8 polypeptide may be utilized to prepare antibodies. See, for example, Kohler et al. (1975) Nature 256:495-497; Kozbor et al. (1985) J. Immunol Methods 81:3142; Cote et al. (1983) Proc Natl Acad Sci 80:2026-2030; and Cole et al. (1984) Mol Cell Biol 62:109-120 for the preparation of monoclonal antibodies; Huse et al. (1989) Science 246:1275-1281 for the preparation of monoclonal Fab fragments; and, Pound (1998) Immunochemical Protocols, Humana Press, Totowa, N.J for the preparation of phagemid or B-lymphocyte immunoglobulin libraries to identify antibodies. The antibodies specific for a kallikrein 8 polypeptide used in the methods of the invention may also be obtained from scientific or commercial sources.

Preferably, antibodies used in the methods of the invention are reactive against a kallikrein 8 polypeptide if they bind with a Kₐ of greater than or equal to 10⁻⁷ M.

An antibody that binds to a kallikrein 8 polypeptide may be labelled with a detectable substance and localised or detected in biological samples based upon the presence of the detectable substance. Examples of detectable substances include, but are not limited to, the following: radioisotopes (e.g., ³H, ¹⁴C, ³⁵S, ¹²⁵I, ¹³¹I), fluorescent labels (e.g., FITC, rhodamine, lanthanide phosphors), luminescent labels such as luminal, enzymatic labels (e.g., horseradish peroxidase, beta-galactosidase, luciferase, alkaline phosphatase, acetylcholinesterase), biotinyl groups (which can be detected by marked avidin e.g., streptavidin containing a fluorescent marker or enzymatic activity that can be detected by optical or calorimetric methods), and predetermined polypeptide epitopes recognized by a secondary reporter (e.g., leucine zipper pair sequences, binding sites for secondary antibodies, metal binding domains, epitope tags). In some embodiments, labels are attached via spacer arms of various lengths to reduce potential steric hindrance. Antibodies may also be coupled to electron dense substances, such as ferritin or colloidal gold, which are readily visualised by electron microscopy.

Indirect methods may also be employed in which the primary antigen-antibody reaction is amplified by the introduction of a second antibody, having specificity for the antibody reactive against kallikrein 8 polypeptide. The second antibody may be labeled with a detectable substance to detect the primary antigen-antibody reaction. By way of example, if the antibody having specificity against a kallikrein 8 polypeptide is a rabbit IgG antibody, the second antibody may be goat anti-rabbit gamma-globulin labelled with a detectable substance as described herein.

Methods for conjugating or labelling the antibodies discussed above may be readily accomplished by one of ordinary skill in the art. (See for example Inman, Methods In Enzymology, Vol. 34, Affinity Techniques, Enzyme Purification: Part B, Jakoby and Wichek (eds.), Academic Press, New York, p. 30, 1974; and Wilchek and Bayer, “The Avidin-Biotin Complex in Bioanalytical Applications,” Anal. Biochem. 171:1-32, 1988 re methods for conjugating or labelling the antibodies with enzyme or ligand binding partner).

Cytochemical techniques known in the art for localizing antigens using light and electron microscopy may be used to detect a kallikrein 8 polypeptide. Generally, an antibody may be labeled with a detectable substance and a kallikrein 8 polypeptide may be localised in tissues and cells based upon the presence of the detectable substance.

In the context of the methods of the invention, the sample, binding agents (e.g. antibodies) for a kallikrein 8 polypeptide may be immobilized on a carrier or support. Examples of suitable carriers or supports are agarose, cellulose, nitrocellulose, dextran, Sephadex, Sepharose, liposomes, carboxymethyl cellulose, polyacrylamides, polystyrene, gabbros, filter paper, magnetite, ion-exchange resin, plastic film, plastic tube, glass, polyamine-methyl vinyl-ether-maleic acid copolymer, amino acid copolymer, ethylene-maleic acid copolymer, nylon, silk, etc. The support material may have any possible configuration including spherical (e.g. bead), cylindrical (e.g. inside surface of a test tube or well, or the external surface of a rod), or flat (e.g. sheet, test strip). Thus, the carrier may be in the shape of, for example, a tube, test plate, well, beads, disc, sphere, etc. The immobilized material may be prepared by reacting the material with a suitable insoluble carrier using known chemical or physical methods, for example, cyanogen bromide coupling. Binding agents (e.g. antibodies) may be indirectly immobilized using second binding agents specific for the first binding agent. For example, mouse antibodies specific for a kallikrein 8 polypeptide may be immobilized using sheep anti-mouse IgG Fc fragment specific antibody coated on the carrier or support.

Where a radioactive label is used as a detectable substance, a kallikrein 8 polypeptide may be localized by radioautography. The results of radioautography may be quantitated by determining the density of particles in the radioautographs by various optical methods, or by counting the grains.

Time-resolved fluorometry may be used to detect a fluorescent signal. For example, the method described in Christopoulos T K and Diamandis E P Anal Chem 1992:64:342-346 may be used with a conventional time-resolved fluorometer.

Therefore, in accordance with an embodiment of the invention, a method is provided wherein a kallikrein 8 specific antibody is labelled with an enzyme, a substrate for the enzyme is added wherein the substrate is selected so that the substrate, or a reaction product of the enzyme and substrate, forms fluorescent complexes with a lanthanide metal. A lanthanide metal is added and kallikrein 8 is quantitated in the sample by measuring fluorescence of the fluorescent complexes. The antibodies specific for kallikrein 8 may be directly or indirectly labelled with an enzyme. Enzymes are selected based on the ability of a substrate of the enzyme, or a reaction product of the enzyme and substrate, to complex with lanthanide metals such as europium and terbium. Examples of enzymes and substrates for enzymes that provide such fluorescent complexes are described in U.S. Pat. No. 5,312,922 to Diamandis. Examples of suitable enzymes include alkaline phosphatase and β-galactosidase. When the antibody is directly or indirectly labelled with alkaline phosphatase the substrate employed in the method may be 4-methylumbelliferyl phosphate, 5-fluorosalicyl phosphate, or diflunisal phosphate. The fluorescence intensity of the complexes is typically measured using a time-resolved fluorometer e.g. a CyberFluor 615 Imunoanalyzer (Nordion International, Kanata, Ontario).

Antibodies specific for a kallikrein 8 polypeptide may also be indirectly labelled with an enzyme. For example, the antibodies may be conjugated to one partner of a ligand binding pair, and the enzyme may be coupled to the other partner of the ligand binding pair. Representative examples include avidin-biotin, and riboflavin-riboflavin binding protein. In an embodiment, the antibodies are biotinylated, and the enzyme is coupled to streptavidin. In another embodiment, antibodies specific for the anti-kallikrein antibodies are labeled with an enzyme.

In accordance with an aspect, the present invention provides means for determining kallikrein 8 polypeptide in a sample by measuring kallikrein 8 polypeptide by immunoassay. It will be evident to a skilled artisan that a variety of immunoassay methods can be used to measure kallikrein 8 polypeptide in a sample in particular a serum sample. In general, a kallikrein 8 polypeptide immunoassay method may be competitive or noncompetitive. Competitive methods typically employ an immobilized or immobilizable antibody to kallikrein 8 polypeptide (anti-K8) and a labeled form of kallikrein 8 polypeptide. Sample kallikrein 8 polypeptide and labeled kallikrein 8 polypeptide compete for binding to anti-K8. After separation of the resulting labeled kallikrein 8 polypeptide that has become bound to anti-K8 (bound fraction) from that which has remained unbound (unbound fraction), the amount of the label in either bound or unbound fraction is measured and may be correlated with the amount of kallikrein 8 polypeptide in the test sample in any conventional manner, e.g. by comparison to a standard curve.

In another aspect, a non-competitive method is used for the determination of a kallikrein 8 polypeptide, with the most common method being the “sandwich” method. In this assay, two anti-K8 antibodies are employed. One of the anti-K8 antibodies is directly or indirectly labeled (sometimes referred to as the “detection antibody”) and the other is immobilized or immobilizable (sometimes referred to as the “capture antibody”). The capture and detection antibodies can be contacted simultaneously or sequentially with the test sample. Sequential methods can be accomplished by incubating the capture antibody with the sample, and adding the detection antibody at a predetermined time thereafter (sometimes referred to as the “forward” method); or the detection antibody can be incubated with the sample first and then the capture antibody added (sometimes referred to as the “reverse” method). After the necessary incubation(s) have occurred, to complete the assay, the capture antibody is separated from the liquid test mixture, and the label is measured in at least a portion of the separated capture antibody phase or the remainder of the liquid test mixture. Generally it is measured in the capture antibody phase since it comprises kallikrein 8 polypeptide bound by (“sandwiched” between) the capture and detection antibodies. In an embodiment, the label may be measured without separating the capture antibodies and liquid test mixture.

In a typical two-site immunometric assay for kallikrein 8 polypeptide, one or both of the capture and detection antibodies are polyclonal antibodies or one or both of the capture and detection antibodies are monoclonal antibodies (i.e. polyclonal/polyclonal, monoclonal/monoclonal, or monoclonal/polyclonal). In a specific embodiment, mouse and rabbit polyclonal antibodies are utilized. The label used in the detection antibody can be selected from any of those known conventionally in the art. The label may be an enzyme or a chemiluminescent moiety, but it can also be a radioactive isotope, a fluorophor, a detectable ligand (e.g., detectable by a secondary binding by a labeled binding partner for the ligand), and the like. Preferably the antibody is labelled with an enzyme which is detected by adding a substrate that is selected so that a reaction product of the enzyme and substrate forms fluorescent complexes. The capture antibody is selected so that it provides a means for being separated from the remainder of the test mixture. Accordingly, the capture antibody can be introduced to the assay in an already immobilized or insoluble form, or can be in a immobilizable form, that is, a form which enables immobilization to be accomplished subsequent to introduction of the capture antibody to the assay. An immobilized capture antibody may comprise an antibody covalently or noncovalently attached to a solid phase such as a magnetic particle, a latex particle, a microtiter plate well, a bead, a cuvette, or other reaction vessel. An example of an immobilizable capture antibody is antibody which has been chemically modified with a ligand moiety, e.g., a hapten, biotin, or the like, and which can be subsequently immobilized by contact with an immobilized form of a binding partner for the ligand, e.g., an antibody, avidin, or the like. In an embodiment, the capture antibody may be immobilized using a species specific antibody for the capture antibody that is bound to the solid phase.

A particular sandwich immunoassay method of the invention employs two antibodies reactive against a kallikrein 8 polypeptide, a second antibody having specificity against an antibody reactive against a kallikrein 8 polypeptide labelled with an enzymatic label, and a fluorogenic substrate for the enzyme. In an embodiment, the enzyme is alkaline phosphatase (ALP) and the substrate is 5-fluorosalicyl phosphate. ALP cleaves phosphate out of the fluorogenic substrate, 5-fluorosalicyl phosphate, to produce 5-fluorosalicylic acid (FSA). 5-Fluorosalicylic acid can then form a highly fluorescent ternary complex of the form FSA-Tb(3+)-EDTA, which can be quantified by measuring the Tb3+fluorescence in a time-resolved mode. Fluorescence intensity is measured using a time-resolved fluorometer as described herein.

The methods described herein may utilize multiple markers for ovarian cancer. Therefore, the invention contemplates a method for anaylzing a biological sample for the presence of kallikrein 8 and other markers that are specific indicators of ovarian cancer. Other markers include markers to kallikreins such as human stratum corneum chymotryptic enzyme (HSCCE), kallikrein 2, kallikrein 3, kallikrein 4, kallikrein 5, kallikrein 6, kallikrein 9, kallikrein 10, and kallikrein 11; CA125, CA15-3, CA72-4, CA19-9, OVX1, lysophosphatidic acid (LPA), creatin-kinase BB, haptoglobin alpha, prostasin, osteopontin, and carcinoembryonic antigen (CEA). Preferably the other markers are markers to kallikreins. In an aspect of the invention, the markers are one or more of kallikrein 6, kallikrein 10, kallikrein 11, and CA125. The methods described herein may be modified by including reagents to detect the markers (e.g. binding agents such as antibodies or nucleic acids specific for the markers).

The above-described immunoassay methods and formats are intended to be exemplary and are not limiting since, in general, it will be understood that any immunoassay method or format can be used in the present invention.

Computer Systems

Computer readable media comprising kallikrein 8 markers is also provided. “Computer readable media” refers to any medium that can be read and accessed directly by a computer, including but not limited to magnetic storage media, such as floppy discs, hard disc storage medium, and magnetic tape; optical storage media such as CD-ROM; electrical storage media such as RAM and ROM; and hybrids of these categories such as magnetic/optical storage media. Thus, the invention contemplates computer readable medium having recorded thereon kallikrein 8 markers identified for patients and controls.

“Recorded” refers to a process for storing information on computer readable medium. The skilled artisan can readily adopt any of the presently known methods for recording information on computer readable medium to generate manufactures comprising information on kallikrein 8 markers.

A variety of data processor programs and formats can be used to store information on kallikrein 8 markers on computer readable medium. For example, the information can be represented in a word processing text file, formatted in commercially-available software such as WordPerfect and MicroSoft Word, or represented in the form of an ASCII file, stored in a database application, such as DB2, Sybase, Oracle, or the like. Any number of dataprocessor structuring formats (e.g., text file or database) may be adapted in order to obtain computer readable medium having recorded thereon the marker information.

By providing the marker information in computer readable form, one can routinely access the information for a variety of purposes. For example, one skilled in the art can use the information in computer readable form to compare marker information obtained during or following therapy with the information stored within the data storage means.

The invention provides a medium for holding instructions for performing a method for determining whether a patient has ovarian cancer or a pre-disposition to ovarian cancer, comprising determining the presence or absence of kallikrein 8 markers, and based on the presence or absence of the kallikrein 8 markers, determining whether the patient has ovarian cancer or a pre-disposition to ovarian cancer, and optionally recommending treatment for the ovarian cancer or pre-ovarian cancer condition.

The invention also provides in an electronic system and/or in a network, a method for determining whether a subject has ovarian cancer or a pre-disposition to ovarian cancer associated with kallikrein markers, comprising determining the presence or absence of kallikrein markers, and based on the presence or absence of the kallikrein markers, determining whether the subject has ovarian cancer or a pre-disposition to ovarian cancer, and optionally recommending treatment for the ovarian cancer or pre-ovarian cancer condition.

The invention further provides in a network, a method for determining whether a subject has ovarian cancer or a pre-disposition to ovarian cancer associated with kallikrein 8 markers, comprising: (a) receiving phenotypic information on the subject and information on kallikrein 8 markers associated with samples from the subject; (b) acquiring information from the network corresponding to the kallikrein 8 markers; and (c) based on the phenotypic information and information on the kallikrein 8 markers determining whether the subject has ovarian cancer or a pre-disposition to ovarian cancer; and (d) optionally recommending treatment for the ovarian cancer or pre-ovarian cancer condition.

The invention still further provides a system for identifying selected records that identify an ovarian cancer cell. A system of the invention generally comprises a digital computer; a database server coupled to the computer; a database coupled to the database server having data stored therein, the data comprising records of data comprising kallikrein 8 markers, and a code mechanism for applying queries based upon a desired selection criteria to the data file in the database to produce reports of records which match the desired selection criteria.

In an aspect of the invention a method is provided for detecting an ovarian cancer cell using a computer having a processor, memory, display, and input/output devices, the method comprising the steps of:
(a) creating records of kallikrein 8 markers isolated from a sample suspected of containing an ovarian cancer cell;
(b) providing a database comprising records of data comprising kallikrein 8 markers; and
(c) using a code mechanism for applying queries based upon a desired selection criteria to the data file in the database to produce reports of records of step (a) which provide a match of the desired selection criteria of the database of step (b) the presence of a match being a positive indication that the markers of step (a) have been isolated from a cell that is an ovarian cancer cell.

The invention contemplates a business method for determining whether a subject has ovarian cancer or a pre-disposition to ovarian cancer associated with kallikrein 8 markers comprising: (a) receiving phenotypic information on the subject and information on kallikrein 8 markers associated with samples from the subject; (b) acquiring information from a network corresponding to the kallikrein 8 markers; and (c) based on the phenotypic information, information on the kallikrein 8 markers, and acquired information, determining whether the subject has ovarian cancer or a pre-disposition to ovarian cancer; and (d) optionally recommending treatment for the ovarian cancer or pre-ovarian cancer condition.

Imaging

Antibodies specific for kallikrein 8 may also be used in imaging methodologies in the management of ovarian cancer. The invention provides a method for imaging tumors associated with one or more kallikreins, preferably kallikreins associated with ovarian cancer, most preferably kallikrein 8 and optionally kallikrein 4, kallikrein 5, kallikrein 6, kallikrein 10, and kallikrein 11.

The invention also contemplates imaging methods described herein using multiple markers for ovarian cancer. For example, a method for imaging ovarian cancer may further comprise injecting the patient with one or more of an imaging agent that binds to human stratum corneum chymotryptic enzyme (HSCCE), kallikrein 2, kallikrein 3, kallikrein 4, kallikrein 5, kallikrein 6, kallikrein 9, kallikrein 10, kallikrein 11, CA125, CA15-3, CA72-4, CA19-9, OVX1, lysophosphatidic acid (LPA), haptoglobin alpha, creatin-kinase BB, osteopontin, prostasin, or carcinoembryonic antigen (CEA), preferably CA 125. Preferably each imaging agent is labeled so that it can be distinguished during the imaging.

In an embodiment the method is an in vivo method and a subject or patient is administered one or more imaging agents that carry an imaging label and that are capable of targeting or binding to a kallikrein.

The imaging agent is allowed to incubate in vivo and bind to the kallikrein(s) associated with a tumor, preferably ovarian tumors. The presence of the label is localized to the ovarian cancer, and the localized label is detected using imaging devices known to those skilled in the art.

An imaging agent may be an antibody or chemical entity which recognizes the kallikrein(s). In an aspect of the invention the imaging agent is a polyclonal antibody or monoclonal antibody, or fragments thereof, or constructs thereof including but not limited to, single chain antibodies, bifunctional antibodies, molecular recognition units, and peptides or entities that mimic peptides. The antibodies specific for the kallikreins used in the methods of the invention may be obtained from scientific or commercial sources, or isolated native kallikrein or recombinant kallikrein may be utilized to prepare antibodies etc as described herein.

An imaging agent may be a peptide that mimics the epitope for an antibody specific for a kallikrein and binds to the kallikrein. The peptide may be produced on a commercial synthesizer using conventional solid phase chemistry. By way of example, a peptide may be prepared that includes either tyrosine, lysine, or phenylalanine to which N₂S₂ chelate is complexed (See U.S. Pat. No. 4,897,255). The anti-kallikrein peptide conjugate is then combined with a radiolabel (e.g. sodium ^{99m}Tc pertechnetate or sodium ¹⁸⁸Re perrhenate) and it may be used to locate a kallikrein producing tumor.

The imaging agent carries a label to image the kallikreins. An imaging agent may be labelled for use in radionuclide imaging. In particular, an imaging agent may be directly or indirectly labelled with a radioisotope. Examples of radioisotopes that may be used in the present invention are the following: ²⁷⁷Ac, ²¹¹At, ¹²⁸Ba, ¹³¹Ba, ⁷Be, ²⁰⁴Bi, ²⁰⁵Bi, ²⁰⁶Bi, ⁷⁶Br, ⁷⁷Br, ⁸²Br, ¹⁰⁹Cd, ⁴⁷Ca, ¹¹C, ¹⁴C, ³⁶Cl, ⁴⁸Cr, ⁵¹Cr, ⁶²Cu, ⁶⁴Cu, ⁶⁷Cu, ¹⁶⁵Dy, ¹⁵⁵Eu, ¹⁸F, ¹⁵³Gd, ⁶⁶Ga, ⁶⁷Ga, ⁶⁸Ga, ⁷²Ga, ¹⁹⁸Au, ³H, ¹⁶⁶Ho, ¹¹¹In, ^{113m}In, ^{115m}In, ¹²³I, ¹²⁵I, ¹³¹I, ¹⁸⁹Ir, ^{191m}Ir, ¹⁹⁴Ir, ⁵²Fe, ⁵⁵Fe, ⁵⁹Fe, ¹⁷⁷Lu, ¹⁵O, ^{191m-191}Os, ¹⁰⁹Pd, ³²P, ³³P, ⁴²K ²²⁶Ra, ¹⁸⁶Re, ¹⁸⁸Re, ^{82m}Rb, ¹⁵³Sm, ⁴⁶Sc, ⁴⁷Sc, ⁷²Se, ⁷⁵Se, ¹⁰⁵Ag, ²²Na, ²⁴Na, ⁸⁹Sr, ³⁵S, ³⁸S, ¹⁷⁷Ta, ⁹⁶Tc, ^{99m}Tc, ²⁰¹Tl, ²⁰²Tl, ¹¹³Sn, ^{117m}Sn, ¹²¹Sn, ¹⁶⁶Yb, ¹⁶⁹Yb, ¹⁷⁵Yb, ⁸⁸Y, ⁹⁰Y, ⁶²Zn and ⁶⁵Zn. Preferably the radioisotope is ¹³¹I, ¹²⁵I, ¹²³I, ¹¹¹I, ^{99m}Tc, ⁹⁰Y, ¹⁸⁶Re, ¹⁸⁸Re, ³²P, ¹⁵³Sm, ⁶⁷Ga, ²⁰¹Tl, ⁷⁷Br, or ¹⁸F, and is imaged with a photoscanning device.

Procedures for labeling biological agents with the radioactive isotopes are generally known in the art. U.S. Pat. No. 4,302,438 describes tritium labeling procedures. Procedures for iodinating, tritium labeling, and ³⁵S labeling especially adapted for murine monoclonal antibodies are described by Goding, J. W. (supra, pp 124-126) and the references cited therein. Other procedures for iodinating biological agents, such as antibodies, binding portions thereof, probes, or ligands, are described in the scientific literature (see Hunter and Greenwood, Nature 144:945 (1962), David et al., Biochemistry 13:1014-1021 (1974), and U.S. Pat. Nos. 3,867,517 and 4,376,110). Iodinating procedures for agents are described by Greenwood, F. et al., Biochem. J. 89:114-123 (1963); Marchalonis, J., Biochem. J. 113:299-305 (1969); and Morrison, M. et al., Immunochemistry, 289-297 (1971). ^{99m} Tc-labeling procedures are described by Rhodes, B. et al. in Burchiel, S. et al. (eds.), Tumor Imaging: The Radioimmunochemical Detection of Cancer, New York: Masson 111-123 (1982) and the references cited therein. Labelling of antibodies or fragments with technetium-99m are also described for example in U.S. Pat. No. 5,317,091, U.S. Pat. No. 4,478,815, U.S. Pat. No. 4,478,818, U.S. Pat. No. 4,472,371, U.S. Pat. No. Re 32,417, and U.S. Pat. No. 4,311,688. Procedures suitable for ¹¹¹In-labeling biological agents are described by Hnatowich, D. J. et al., J. Immul. Methods, 65:147-157 (1983), Hnatowich, D. et al., J. Applied Radiation, 35:554-557 (1984), and Buckley, R. G. et al., F.E.B.S. 166:202-204 (1984).

An imaging agent may also be labeled with a paramagnetic isotope for purposes of an in vivo method of the invention. Examples of elements that are useful in magnetic resonance imaging include gadolinium, terbium, tin, iron, or isotopes thereof. (See, for example, Schaefer et al., (1989) JACC 14, 472-480; Shreve et al., (1986) Magn. Reson. Med. 3, 336-340; Wolf, G L., (1984) Physiol. Chem. Phys. Med. NMR 16, 93-95; Wesbey et al., (1984) Physiol. Chem. Phys. Med. NMR 16, 145-155; Runge et al., (1984) Invest. Radiol. 19, 408415 for discussions on in vivo nuclear magnetic resonance imaging.)

In the case of a radiolabeled imaging agent, the agent may be administered to the patient, it is localized to the tumor having a kallikrein with which the agent binds, and is detected or “imaged” in vivo using known techniques such as radionuclear scanning using e.g., a gamma camera or emission tomography. [See for example, A. R. Bradwell et al., “Developments in Antibody Imaging”, Monoclonal Antibodies for Cancer Detection and Therapy, R. W. Baldwin et al., (eds.), pp. 65-85 (Academic Press 1985)]. A positron emission transaxial tomography scanner, such as designated Pet VI located at Brookhaven National Laboratory, can also be used where the radiolabel emits positrons (e.g., ¹¹C, ¹⁸F, ¹⁵O, and ¹³N).

Whole body imaging techniques using radioisotope labeled agents can be used for locating both primary tumors and tumors which have metastasized. Antibodies specific for kallikreins, or fragments thereof having the same epitope specificity, are bound to a suitable radioisotope, or a combination thereof, and administered parenterally. For ovarian cancer, administration preferably is intravenous. The bio-distribution of the label can be monitored by scintigraphy, and accumulations of the label are related to the presence of ovarian cancer cells. Whole body imaging techniques are described in U.S. Pat. Nos. 4,036,945 and 4,311,688. Other examples of agents useful for diagnosis and therapeutic use which can be coupled to antibodies and antibody fragments include metallothionein and fragments (see, U.S. Pat. No. 4,732,864). These agents are useful in diagnosis, staging and visualization of cancer, in particular ovarian cancer, so that surgical and/or radiation treatment protocols can be used more efficiently.

Screening Methods

The invention also contemplates methods for evaluating test agents or compounds for their ability to inhibit ovarian cancer or potentially contribute to ovarian cancer. Test agents and compounds include but are not limited to peptides such as soluble peptides including Ig-tailed fusion peptides, members of random peptide libraries and combinatorial chemistry-derived molecular libraries made of D- and/or L-configuration amino acids, phosphopeptides (including members of random or partially degenerate, directed phosphopeptide libraries), antibodies [e.g. polyclonal, monoclonal, humanized, anti-idiotypic, chimeric, single chain antibodies, fragments, (e.g. Fab, F(ab)₂, and Fab expression library fragments, and epitope-binding fragments thereof)], and small organic or inorganic molecules. The agents or compounds may be endogenous physiological compounds or natural or synthetic compounds.

In an aspect, the invention provides a method for assessing the potential efficacy of a test agent for inhibiting ovarian cancer in a patient, the method comprising comparing:
(a) levels of kallikrein 8 markers in a first sample obtained from a patient and exposed to the test agent, and
(b) levels of the kallikrein 8 markers in a second sample obtained from the patient, wherein the sample is not exposed to the test agent, wherein a significant difference in the levels of expression of kallikrein 8 markers in the first sample, relative to the second sample, is an indication that the test agent is potentially efficacious for inhibiting ovarian cancer in the patient.

The first and second samples may be portions of a single sample obtained from a patient or portions of pooled samples obtained from a patient.

In another aspect, the invention provides a method of selecting an agent for inhibiting ovarian cancer in a patient comprising:
(a) obtaining a sample comprising ovarian cancer cells from the patient;
(b) separately maintaining aliquots of the sample in the presence of a plurality of test agents;
(c) comparing kallikrein 8 markers in each of the aliquots; and
(d) selecting one of the test agents which alters the levels of the kallikrein 8 markers in the aliquot containing that test agent, relative to other test agents.

Still another aspect of the present invention provides a method of conducting a drug discovery business comprising:
(a) providing one or more methods or assay systems of the invention for identifying agents that inhibit ovarian cancer in a patient;
(b) conducting therapeutic profiling of agents identified in step (a), or further analogs thereof, for efficacy and toxicity in animals; and
(c) formulating a pharmaceutical preparation including one or more agents identified in step (b) as having an acceptable therapeutic profile.

In certain embodiments, the subject method can also include a step of establishing a distribution system for distributing the pharmaceutical preparation for sale, and may optionally include establishing a sales group for marketing the pharmaceutical preparation.

The invention also contemplates a method of assessing the ovarian cancer carcinogenic potential of a test compound comprising:
(a) maintaining separate aliquots of ovarian cancer cells in the presence and absence of the test compound; and
(b) comparing kallikrein 8 markers encoding same in each of the aliquots.

A significant difference between the levels of the markers in the aliquot maintained in the presence of (or exposed to) the test compound relative to the aliquot maintained in the absence of the test compound, indicates that the test compound possesses ovarian cancer carcinogenic potential.

Kits

The invention contemplates kits for carrying out the methods of the invention. Such kits typically comprise two or more components required for performing a diagnostic assay. Components include but are not limited to compounds, reagents, containers, and/or equipment.

In an aspect of the invention, a container with a kit comprises binding agents as described herein. In particular, the invention may provide a pre-packaged diagnostic kit comprising a binding agent described herein which may be conveniently used, e.g. in clinical settings, to screen and diagnose patients, and to screen and identify those subjects afflicted with or exhibiting a predisposition to ovarian cancer. In an embodiment, the kit may contain antibodies specific for a kallikrein 8 polypeptide, antibodies against the antibodies labelled with enzymes, and substrates for the enzymes. The kit may also contain microtiter plate wells, standards, assay diluent, wash buffer, adhesive plate covers, and/or instructions for carrying out a method of the invention using the kit.

In an aspect of the invention, the kit includes antibodies or antibody fragments which bind specifically to epitopes of a kallikrein 8 polypeptide, and means for detecting binding of the antibodies to epitopes associated with tumor cells, either as concentrates (including lyophilized compositions), which may be further diluted prior to use or at the concentration of use, where the vials may include one or more dosages. Where the kits are intended for in vivo use, single dosages may be provided in sterilized containers, having the desired amount and concentration of agents. Containers that provide a formulation for direct use, usually do not require other reagents, as for example, where the kit contains radiolabelled antibody preparations for in vivo imaging.

The reagents suitable for applying the screening methods of the invention to evaluate compounds may be packaged into convenient kits described herein providing the necessary materials packaged into suitable containers.

Therapeutic Applications

Kallikrein 8 polypeptides are targets for ovarian cancer immunotherapy. Such immunotherapeutic methods include the use of antibody therapy, in vivo vaccines, and ex vivo immunotherapy approaches.

In one aspect, the invention provides antibodies specific for kallikrein 8 polypeptides that may be used systemically to treat ovarian cancer. Preferably antibodies are used that target the tumor cells but not the surrounding non-tumor cells and tissue. Thus, the invention provides a method of treating a patient susceptible to, or having a cancer that expresses a kallikrein 8 polypeptide comprising administering to the patient an effective amount of antibodies that bind specifically to a kallikrein 8 polypeptide. In another aspect, the invention provides a method of inhibiting the growth of tumor cells expressing a kallikrein 8 polypeptide, comprising administering to a patient antibodies which bind specifically to a kallikrein 8 polypeptide in amounts effective to inhibit growth of the tumor cells. Antibodies specific for a kallikrein 8 polypeptide may also be used in a method for selectively inhibiting the growth of, or killing a cell expressing a kallikrein 8 polypeptide comprising reacting antibody immunoconjugates or immunotoxins with the cell in an amount sufficient to inhibit the growth of, or kill the cell.

By way of example, unconjugated antibodies specific for a kallikrein 8 polypeptide may be introduced into a patient such that the antibodies bind to cancer cells expressing a kallikrein 8 polypeptide and mediate growth inhibition of such cells (including the destruction thereof), and the tumor, by mechanisms which may include complement-mediated cytolysis, antibody-dependent cellular cytotoxicity, altering the physiologic function of a kallikrein 8 polypeptide and/or the inhibition of ligand binding or signal transduction pathways. In addition to unconjugated antibodies, antibodies specific for a kallikrein 8 polypeptide, conjugated to therapeutic agents (e.g. immunoconjugates) may also be used therapeutically to deliver the agents directly to tumor cells expressing a kallikrein 8 polypeptide and thereby destroy the tumor. Examples of such agents include abrin, ricin A, Pseudomonas exotoxin, or diphtheria toxin, proteins such as tumor necrosis factor, alpha-interferon, beta-interferon, nerve growth factor, platelet derived growth factor, tissue plasminogen activator, and biological response modifiers such as lymphokines, interleukin-1, interleukin-2, interleukin-6, granulocyte macrophage colony stimulating factor, granulocyte colony stimulating factor, or other growth factors.

Cancer immunotherapy using antibodies specific for a kallikrein 8 polypeptide may utilize the various approaches that have been successfully employed for cancers, including but not limited to colon cancer (Arlen et al., 1998, Crit Rev Immunol 18: 133-138), multiple myeloma (Ozaki et al., 1997, Blood 90: 3179-3186; Tsunenati et al., 1997, Blood 90: 2437-2444), gastric cancer (Kasprzyk et al., 1992, Cancer Res 52: 2771-2776), B-cell lymphoma (Funakoshi et al., 1996, J Immunther Emphasis Tumor Immunol 19: 93-101), leukemia (Zhong et al., 1996, Leuk Res 20: 581-589), colorectal cancer (Moun et al., 1994, Cancer Res 54: 6160-6166); Velders et al., 1995, Cancer Res 55: 4398-4403), and breast cancer (Shepard et al., 1991, J Clin Immunol 11: 117-127).

In the practice of a method of the invention, antibodies specific for a kallikrein 8 polypeptide capable of inhibiting the growth of cancer cells expressing a kallikrein 8 polypeptide are administered in a therapeutically effective amount to cancer patients whose tumors express or overexpress a kallikrein 8 polypeptide. The invention may provide a specific, effective and long-needed treatment for ovarian cancer. The antibody therapy methods of the invention may be combined with other therapies including chemotherapy and radiation.

Patients may be evaluated for the presence and levels of kallikrein 8 polypeptide expression and overexpression in tumors, preferably using immunohistochemical assessments of tumor tissue, quantitative imaging as described herein, or other techniques capable of reliably indicating the presence and degree of expression of kallikrein 8 polypeptides. Immunohistochemical analysis of tumor biopsies or surgical specimens may be employed for this purpose.

Antibodies specific for kallikrein 8 polypeptides useful in treating cancer include those that are capable of initiating a potent immune response against the tumor and those that are capable of direct cytotoxicity. In this regard, the antibodies may elicit tumor cell lysis by either complement-mediated or antibody-dependent cell cytotoxicity (ADCC) mechanisms, both of which require an intact Fc portion of the immunoglobulin molecule for interaction with effector cell Fc receptor sites or complement proteins. In addition, antibodies specific for kallikrein 8 polypeptides that exert a direct biological effect on tumor growth are useful in the practice of the invention. Such antibodies may not require the complete immunoglobulin to exert the effect. Potential mechanisms by which such directly cytotoxic antibodies may act include inhibition of cell growth, modulation of cellular differentiation, modulation of tumor angiogenesis factor profiles, and the induction of apoptosis. The mechanism by which a particular antibody exerts an anti-tumor effect may be evaluated using any number of in vitro assays designed to determine ADCC, antibody-dependent macrophage-mediated cytotoxicity (ADMMC), complement-mediated cell lysis, and others known in the art.

The anti-tumor activity of antibodies specific for kallikrein 8 polypeptides may be evaluated in vivo using a suitable animal model. Xenogenic cancer models, wherein human cancer explants or passaged xenograft tissues are introduced into immune compromised animals, such as nude or SCID mice, may be employed.

The methods of the invention contemplate the administration of combinations, or “cocktails” of different individual antibodies recognizing epitopes of kallikrein 8 polypeptides and optionally other markers of ovarian cancer (e.g. other kallikreins, CA125, etc). Such cocktails may have certain advantages inasmuch as they contain antibodies that bind to different epitopes and/or exploit different effector mechanisms or combine directly cytotoxic antibodies with antibodies that rely on immune effector functionality. Such antibodies in combination may exhibit synergistic therapeutic effects. In addition, the administration of the antibodies may be combined with other therapeutic agents, including but not limited to chemotherapeutic agents, androgen-blockers, and immune modulators (e.g., IL2, GM-CSF). The antibodies may be administered in their “naked” or unconjugated form, or may have therapeutic agents conjugated to them.

The antibodies specific for kallikrein 8 polypeptides used in the practice of the methods of the invention may be formulated into pharmaceutical compositions comprising a carrier suitable for the desired delivery method. Suitable carriers include any material which when combined with the antibodies retains the anti-tumor function of the antibodies and is non-reactive with the subjects immune systems. Examples include any of a number of standard pharmaceutical carriers such as sterile phosphate buffered saline solutions, bacteriostatic water, and the like (see, generally, Remington's Pharmaceutical Sciences, Mack Publishing Company, Easton, Pa., USA).

Antibody formulations may be administered via any route capable of delivering the antibodies to the tumor site. Routes of administration include, but are not limited to, intravenous, intraperitoneal, intramuscular, intratumor, intradermal, and the like. Preferably, the route of administration is by intravenous injection. Antibody preparations may be lyophilized and stored as a sterile powder, preferably under vacuum, and then reconstituted in bacteriostatic water containing, for example, benzyl alcohol preservative, or in sterile water prior to injection.

Treatment will generally involve the repeated administration of the antibody preparation via an acceptable route of administration such as intravenous injection (IV), at an effective dose. Dosages will depend upon various factors generally appreciated by those of skill in the art, including the type of cancer and the severity, grade, or stage of the cancer, the binding affinity and half life of the antibodies used, the degree of expression of kallikrein 8 polypeptides in the patient, the extent of circulating kallikrein 8 polypeptide antigens the desired steady-state antibody concentration level, frequency of treatment, and the influence of any chemotherapeutic agents used in combination with a treatment method of the invention.

Daily doses may range from about 0.1 to 100 mg/kg. Doses in the range of 10-500 mg antibodies per week may be effective and well tolerated, although even higher weekly doses may be appropriate and/or well tolerated. A determining factor in defining the appropriate dose is the amount of antibodies necessary to be therapeutically effective in a particular context. Repeated administrations may be required to achieve tumor inhibition or regression. Direct administration of antibodies specific for kallikrein 8 polypeptides is also possible and may have advantages in certain situations.

Patients may be evaluated for kallikrein 8 polypeptides, preferably in serum, in order to assist in the determination of the most effective dosing regimen and related factors. The assay methods described herein, or similar assays, may be used for quantitating circulating kallikrein 8 polypeptide levels in patients prior to treatment. Such assays may also be used for monitoring throughout therapy, and may be useful to gauge therapeutic success in combination with evaluating other parameters, such as serum kallikrein 8 polypeptides.

The invention further provides vaccines formulated to contain kallikrein 8 polypeptides or fragments thereof. The use in anti-cancer therapy of tumor antigens in a vaccine for generating humoral and cell-mediated immunity is well known and, for example, has been employed in ovarian cancer using human PSMA and rodent PAP immunogens (Hodge et al., 1995, Int. J. Cancer 63: 231-237; Fong et al., 1997, J. Immunol. 159: 3113-3117). These methods can be practiced by employing kallikrein 8 polypeptides, or fragments thereof, or nucleic acids and recombinant vectors capable of expressing and appropriately presenting the kallikrein 8 immunogens.

By way of example, viral gene delivery systems may be used to deliver nucleic acids encoding kallikrein 8 polypeptides. Various viral gene delivery systems which can be used in the practice of this aspect of the invention include, but are not limited to, vaccinia, fowlpox, canarypox, adenovirus, influenza, poliovirus, adeno-associated virus, lentivirus, and sindbus virus (Restifo, 1996, Curr. Opin. Immunol. 8: 658-663). Non-viral delivery systems may also be employed by using naked DNA encoding kallikrein 8 polypeptides, or fragments thereof introduced into the patient (e.g., intramuscularly) to induce an anti-tumor response.

Various ex vivo strategies may also be employed. One approach involves the use of cells to present kallikrein 8 antigens to a patients immune system. For example, autologous dendritic cells which express MHC class I and II, may be pulsed with kallikrein 8 polypeptides, or peptides thereof that are capable of binding to MHC molecules, to thereby stimulate ovarian cancer patients' immune systems (See, for example, Tjoa et al., 1996, Ovarian 28: 65-69; Murphy et al., 1996, Ovarian 29: 371-380).

Anti-idiotypic antibodies specific for kallikrein 8 polypeptides can also be used in anti-cancer therapy as a vaccine for inducing an immune response to cells expressing the polypeptides. The generation of anti-idiotypic antibodies is well known in the art and can readily be adapted to generate anti-idiotypic antibodies that mimic an epitope on a kallikrein 8 polypeptide (see, for example, Wagner et al., 1997, Hybridoma 16: 3340; Foon et al., 1995, J Clin Invest 96: 334-342; Herlyn et al., 1996, Cancer Immunol Immunother 43: 65-76). Such antibodies can be used in anti-idiotypic therapy as presently practiced with other anti-idiotypic antibodies directed against tumor antigens.

Genetic immunization methods may be utilized to generate prophylactic or therapeutic humoral and cellular immune responses directed against cancer cells expressing one or more kallikrein 8 polypeptides. Constructs comprising DNA encoding kallikrein 8 polypeptides/immunogens and appropriate regulatory sequences may be injected directly into muscle or skin of an individual, such that the cells of the muscle or skin take-up the construct and express the encoded kallikrein 8 polypeptides/immunogens. The polypeptides/immunogens may be expressed as cell surface proteins or be secreted. Expression of the polypeptides/immunogens results in the generation of prophylactic or therapeutic humoral and cellular immunity against the cancer. Various prophylactic and therapeutic genetic immunization techniques known in the art may be used.

The invention further provides methods for inhibiting cellular activity (e.g., cell proliferation, activation, or propagation) of a cell expressing a kallikrein 8 polypeptide. This method comprises reacting immunoconjugates of the invention (e.g., a heterogeneous or homogenous mixture) with the cell so that the kallikrein 8 polypeptide forms a complex with the immunoconjugates. A subject with a neoplastic or preneoplastic condition can be treated when the inhibition of cellular activity results in cell death.

In another aspect, the invention provides methods for selectively inhibiting a cell expressing a kallikrein 8 polypeptide by reacting an immunoconjugate of the invention with the cell in an amount sufficient to inhibit the cell. Amounts include those that are sufficient to kill the cell or sufficient to inhibit cell growth or proliferation.

Kallikrein 8 polypeptides including fragments thereof, or agents identified using a method of the invention may be used in the treatment of ovarian cancer in a subject. The kallikrein 8 polypeptides and agents may be formulated into compositions for administration to subjects suffering from ovarian cancer. Therefore, the present invention also relates to a composition comprising a kallikrein 8 polypeptide including a fragment thereof, or an agent identified using a method of the invention, and a pharmaceutically acceptable carrier, excipient or diluent. A method for treating or preventing ovarian cancer in a subject is also provided comprising administering to a patient in need thereof a kallikrein 8 polypeptide or an agent identified in accordance with a method of the invention, or a composition of the invention.

The active substance may be administered in a convenient manner such as by injection (subcutaneous, intravenous, etc.), oral administration, inhalation, transdermal application, or rectal administration. Depending on the route of administration, the active substance may be coated in a material to protect the substance from the action of enzymes, acids and other natural conditions that may inactivate the substance.

The compositions described herein can be prepared by per se known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985). On this basis, the compositions include, albeit not exclusively, solutions of the active substances in association with one or more pharmaceutically acceptable vehicles or diluents, and contained in buffered solutions with a suitable pH and iso-osmotic with the physiological fluids.

The compositions are indicated as therapeutic agents either alone or in conjunction with other therapeutic agents or other forms of treatment (e.g. chemotherapy or radiotherapy). The compositions of the invention may be administered concurrently, separately, or sequentially with other therapeutic agents or therapies.

The following non-limiting examples are illustrative of the present invention:

### EXAMPLE 1

Materials and Methods

Materials and Methods

Cloning of full-length human kallikrein 8 (KLK8) into baculovirus. The 887-base fragment containing the full-length KLK8 cDNA was cut with EcoRI restriction enzyme from the KLK8/pGEM-T Easy plasmid (9) and ligated into the EcoRI sites of the pVL1393 transfer vector (Pharmingen, Mississauga, Canada) to create plasmid KLK8/pVL1393. This plasmid was transferred into the Autographa californica nuclear polyhedrosis virus (AcNPV) genome by homologous recombination so that High Five™ insect cells were transfected with the transfer vector and AcNPV DNA. The baculovirus containing the full-length KLK8 cDNA was amplified for hK8 protein production as described below.

Protein production. High Five insect cells were cultured in polystyrene flasks (75-cm²) with 25 mL of TNM-FH complete medium (Pharmingen) at 27° C. until almost confluent. Medium in each flask was then changed with 25 mL of serum-free medium (Invitrogen, Burlington, Canada). The cells were then infected with 100 μL of stock baculovirus solution containing full-length KLK8 cDNA (1.3×10⁸-pfu/mL) and cultured at 27° C. for 4 days. The extracellular medium in each flask was harvested and dialyzed against 10 mmol/L HEPES buffer (pH 7.4) at 4° C. for 3 days.

Protein purification. Recombinant hK8 in the dialyzed medium was purified using a three-step column chromatography procedure. The medium was first applied onto a cation exchange column, HiTrap SP Sepharose HP (bed volume: 5 mL, Amersham Pharmacia Biotech, Baie d'Urfe, Canada) and equilibrated with 10 mmol/L HBEPES buffer (pH 7.4). Proteins were eluted with each 10 mL of 10 mmol/L HBEPES buffer (pH 7.4) containing 50, 100 and 150 mmol/L NaCl. One mL fractions were separately collected in tubes. The hK8 content of fractions was determined by Western blotting with a rabbit antiserum as described elsewhere (17). See below for details. Fractions containing hK8 were diluted 1:2 with 10 mmol/L HEPES buffer (pH 7.4) and applied onto an affinity column, HiTrap Heparin HP (bed volume: 5 mL, Amersham Pharmacia Biotech), equilibrated with 10 mmol/L HEPES buffer (pH 7.4). Proteins were eluted with each 10 mL of 10 mmol/L HBEPES buffer (pH 7.4) containing 200 and 250 mmol/L NaCl two-step gradients and collected as 1 mL fractions. Fractions containing hK8 were then applied onto another affinity column, HiTrap Benzamidine FF (bed volume: 1 mL, Amersham Pharmacia Biotech) and equilibrated with 10 mmol/L HEPES buffer (pH 7.4) containing 500 mmol/L NaCl. The flow-through from the benzamidine column was collected. At each step of purification, all fractions were monitored with a sodium dodecyl sulfate polyacrylamide gel electrophoresis (SDS-PAGE) followed by a Coomassie G-250 staining and Western blotting. Each fraction was evaluated with the NuPAGE Bis-Tris electrophoresis system using two 4-12% gradient polyacrylamide gels (Invitrogen). One gel was stained with a Coomassie G-250 staining solution, SimplyBlue SafeStain (Invitrogen). The proteins on the other gel were transferred to a nitrocellulose membrane (Amersham Pharmacia Biotech). After blocking with 5% skim milk in 0.1 mol/L Tris-HCl buffer (pH 7.5) containing 0.15 mol/L NaCl and 0.1% Tween 20 (TBST) for 30 minutes at room temperature, the membranes were reacted with a rabbit anti-hK8 antiserum (17), and further reacted with goat anti-rabbit IgG conjugated with alkaline phosphatase (Jackson ImmunoResearch Inc., West Grove, Pa.) in 5% skim milk-TBST. The secondary antibody was detected on an X-ray film by a chemiluminescent substrate (Diagnostic Products Corporation, Los Angeles, Calif.). The band intensities on the film were analyzed using an image analysis software (Lab Works; Ultra-Violet Products Ltd., Cambridge, UK).

Mass spectrometry. Positive identification and characterization of the recombinant hK8 protein was achieved by trypsin digestion and nanoelectrospray mass spectroscopy, as previously described in detail for recombinant hK10 (18).

Production of polyclonal antibodies. The purified hK8 was used as an immunogen to immunize rabbits and mice. The protein solution containing 100 μg of hK8 (in 150 μL solution for mice or in 400 μL solution for rabbits) was mixed with the same volume of complete Freund's adjuvant for the first injection and incomplete Freund's adjuvant for the subsequent injections. The mixed solution was injected subcutaneously into female Balb/c mice and New Zealand white rabbits. Injections were repeated three times for mice and six times for rabbits at three-week intervals. Blood was drawn from the animals and tested for antibody generation. To test for production of anti-hK8 polygonal antibodies, the following immunoassay was used. Sheep anti-mouse or goat anti-rabbit IgG (Jackson ImmunoResearch) was immobilized on 96-well white ELISA plates. The mouse/rabbit serum was applied to the plates at different dilutions ranging from 1:500 to 1:50,000. After incubation and washing, biotinylated recombinant hK8 was added (5-10 ng/well). Finally, after incubation and washing, alkaline phosphatase-conjugated streptavidin was added, and the alkaline phosphatase activity was detected with time-resolved fluorescence as described in detail elsewhere (19).

Standard immunoassay procedure. White polystyrene microtiter plates were coated with sheep anti-mouse IgG, Fc fragment-specific antibody (Jackson ImmunoResearch). One hundred μL of coating antibody solution [50 mmol/L Tris-HCl buffer (pH 7.8) containing 5 mg/L antibody] was applied in each well and incubated overnight. The plates were washed four times with the washing buffer [10 mmol/L Tris-HCl buffer (pH 7.4) containing 150 mmol/L NaCl and 0.05% Tween 20]. Mouse anti-hK8 antiserum was diluted 2,000-fold in a general diluent [50 mmol/L Tris-HCl buffer (pH 7.8) containing 6% bovine serum albumin (BSA) and 0.05% sodium azide], and 100 μL of the diluted antiserum was applied to each well. After 2 hours incubation with shaking, the plates were washed six times with the washing buffer. hK8 standards or samples were applied in each well (50 μL/well) along with 50 μL of general diluent and incubated for 2 hours with shaking. The plates were washed with washing buffer six times. Subsequently, 100 μL of rabbit anti-hK8 antiserum diluted 1,000-fold in buffer A [50 mmol/L Tris-HCl buffer (pH 7.8) containing 0.5 mol/L KCl, 6% BSA, 5% goat serum, 2.5% mouse serum, 1% bovine globulin and 0.5% Tween 20] was applied to each well and incubated for 1 hour. Plates were washed as above. Finally, 100 μL/well of alkaline phosphatase-conjugated goat anti-rabbit IgG, Fc fragment-specific (Jackson ImmunoResearch), diluted 3,000-fold in buffer A was added to each well and incubated for 1 hour, and plates were washed as above. One hundred μL of diflunisal phosphate (DFP) solution [0.1 mol/L Tris-HCl buffer (pH 9.1) containing 1 mmol/L DFP, 0.1 mol/L NaCl and 1 mmol/L MgCl₂l] was added to each well and incubated for 10 minutes. One hundred μL of developing solution (1 mol/L Tris base, 0.4 mol/L NaOH, 2 mmol/L TbCl₃ and 3 mmol/L EDTA) was applied into each well and mixed for 1 minute. The fluorescence was measured with a time-resolved fluorometer, the Cyberfluor 615 Immunoanalyzer (MDS Nordion, Kanata, Canada). The calibration and data reduction were performed automatically, as described in detail elsewhere(18;19).

Sensitivity of the immunoassay. Recombinant hK8 was used to generate the calibration curve. hK8 standards were prepared by diluting the purified recombinant hK8 in the general diluent. These calibrators were used to define the detection limit of the assay.

Preparation of human tissue extracts and biological fluids. The following human tissues (adult and fetal) were used for screening: Esophagus, tonsil, skin, testis, kidney, salivary gland, breast, fallopian tube, adrenal, bone, cerebellum, colon, endometrium, liver, lung, muscle, ovary, pancreas, pituitary, prostate, seminal vesicle, small intestine, spinal cord, spleen, stomach, thyroid, trachea and ureter. Human tissue extracts were prepared as follows: Frozen human tissues (0.2 g) were pulverized on dry ice to fine powders. Two mL of extraction buffer [50 mmol/L Tris-HCl buffer (pH 8.0) containing 150 mmol/L NaCl, 5 mmol/L EDTA, 1% NP-40] surfactant was added to the tissue powders and the mixture was incubated on ice for 30 minutes with repeated shaking and vortex-mixing every 10 minutes. Mixtures were centrifuged at 14,000×g at 4° C. for 30 minutes. The supernatants were collected as tissue extracts and stored at −80° C. until just before use. Ovarian and breast cancer cytosols previously prepared for steroid hormone receptor analysis as previously described (20) were also tested. The biological fluids (amniotic fluid, breast milk, cerebrospinal fluid, follicular fluid, serum, seminal plasma and ascites fluid from women with advanced ovarian cancer) were leftovers of samples submitted for routine biochemical testing. Biological fluids were stored at −80° C. until use.

Specificity of the immunoassay. Amniotic fluid, esophagus extract, tonsil extract and recombinant hK8 were used to confirm the specificity of the developed immunoassay. These samples were measured by the standard assay procedure described above. The mouse and rabbit anti-hK8 antisera were then successively replaced with sera from the same animals obtained before immunization (preimmune sera). The samples were then measured again, and the fluorescence counts were compared with the counts obtained by the standard assay. The cross-reactivities of other homologous proteins were also investigated using recombinant hK2 (250 μg/L) and recombinant hK3, hK4, hK5, hK6, hK7, hK9, hK10, hK11, hK12 and hK13, all at a concentration of 1,000 μg/L. Recombinant hK8 (100 μg/L) and these reference samples were measured with the standard procedure described above; their fluorescence counts were then compared.

Linearity. To determine the linearity of the hK8 immunoassay, milk and amniotic fluid samples were serially diluted in general diluent, and their hK8 concentrations were measured with the standard assay.

Recovery. Recombinant hK8 was added to breast cytosols, seminal plasma and normal sera (male and female) at different concentrations and measured with the developed hK8 immunoassay. Recoveries were calculated after subtraction of the endogenous concentrations.

Fractionation of Biological Fluids with gel filtration HPLC To determine the molecular mass of the protein detected in the biological fluids and tissue extracts, amniotic fluid, ascites fluid, breast milk and ovarian cancer serum were fractionated with gel filtration chromatography, as described elsewhere (21). The fractions were then collected and analyzed for hK8 using the developed immunoassay.

Cancer cell lines and hormonal stimulation experiments. The breast cancer cell lines MDA-MB-231, BT-474, T-47D, MCF-7, and ZR-75, the ovarian cancer cell line HTB-75 (Caov-3) and the prostate cancer cell line LNCaP were purchased from the American Type Culture Collection (ATCC), Rockville, Md. The BG-1 ovarian cancer cell line was kindly provided by Dr. Henri Rochefort, Montpellier, France and the PC-3 cell line, stably transfected with androgen receptor [PC-3 (AR)₆] was provided by Dr. Theodore Brown, Toronto, Canada. Cells were cultured in RPMI media (Invitrogen) supplement with glutamine (200 nmol/L) and fetal bovine serum (10%), in plastic flasks, to near confluency. The cells were then aliquoted into 24-well tissue culture plates and cultured to 50% confluency. Twenty-four hours before the experiments, the culture medium was changed into a medium containing 10% charcoal-stripped fetal bovine serum. For stimulation experiments, various steroid hormones dissolved in 100% ethanol were added into the culture media, at a final concentration of 10⁻⁸ mol/L. Steroids tested were aldosterone (mineralocorticoid), dexamethasone (glucocorticoid), norgestrel (androgenic progestin), dihydrotestosterone (androgen) and estradiol (estrogen). Cells stimulated with 100% ethanol were included as controls. The cells were grown for 7 days and the cell cultured supernatants were collected for hK8 examination with the developed immunoassay. These experiments were repeated at least twice.

Statistical analysis. Statistical analysis was performed with SAS software (SAS Institute, Cary, N.C.). All data were analyzed with non-parametric tests and relationships between different variables were assessed by Spearman correlation. Survival analysis was performed by using Kaplan-Meier plots and the differences between curves were evaluated by the log-rank test. A p value of <0.05 was considered statistically significant.

Results

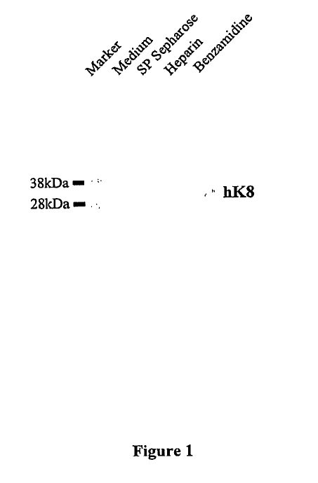

Production and purification of hK8 protein. The 31-kDa recombinant hK8 was expressed and secreted into the medium of baculovirus-infected High Five insect cells. The hK8 in the medium was detected by Western blot analysis 1 day after infection and the strongest signal was detected at 4 days. Almost purified hK8 after SP Sepharose, heparin, and benzamidine purification steps was detected in 100 mmol/L NaCl eluent, 200 mmol/L NaCl eluent and flow-through fractions, respectively. One hundred and eight μg of purified hK8 was obtained with the three-step column chromatography purification procedure from 400 mL of culture medium (Table 1). Finally, purified hK8 was detected as a single band on a 4-12% SDS-PAGE gel stained with Coomassie G-250 solution (FIG. 1). This band was subjected to MALDI-TOF and MS/MS mass spectrometric analysis as described elsewhere (18) and confirmed to be human hK8 (data not shown).

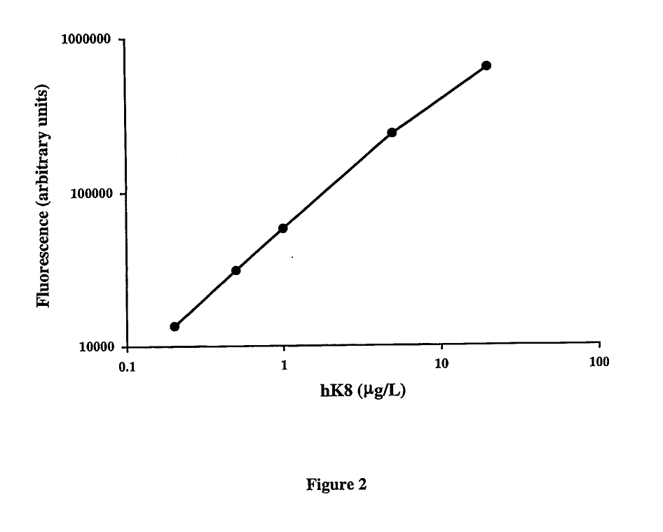

Standard immunoassay procedure. A typical calibration curve for the hK8 immunofluorometric procedure is shown in FIG. 2. The detection limit, defined as the concentration of analyte that can be distinguished from zero with 95% confidence, was 0.2 μg/L. The dynamic range extends to 20 μg/L. The within-run and day-to-day coefficients of variation (CVs) for the developed hK8 assay were <10% within the measurement range, consistent with the precision of typical microtiter plate-based immunoassays.

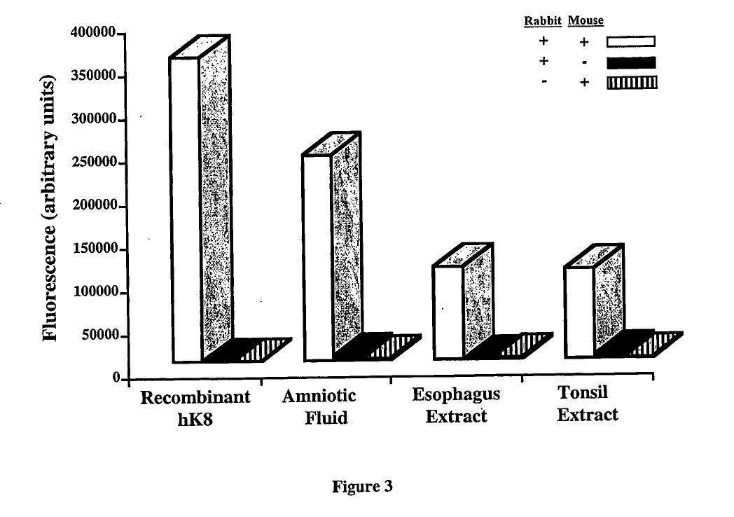

Specificity. When amniotic fluid, esophagus extract, tonsil extract, and recombinant hK8 (100 μg/L) were measured with the developed assay, fluorescence counts>100,000 arbitrary units were obtained (FIG. 3). However, when either mouse or rabbit anti-hK8 polyclonal antibody was replaced with preimmune serum, fluorescence counts were reduced to background signals (<2,000 arbitrary units). No immunoreactivity was detected when hK2, hK3, hK4, hK5, hK6, hK7, hK9, hK10, hK11, hK12 and hK13 solutions (all 1,000 μg/L except hK2, 250 μg/L) were measured with the developed assay for hK8. This data suggest that the assay is very specific for hK8 protein. Breast milk and amniotic fluid samples diluted linearly, with obtained values within 10% of expected values from the undiluted samples, suggesting freedom from matrix effects.

Recovery. Recoveries of added recombinant hK8 were 95-100% in breast cytosols, 97-100% in seminal plasmas, 51-62% in male sera and 36-78% in female sera.

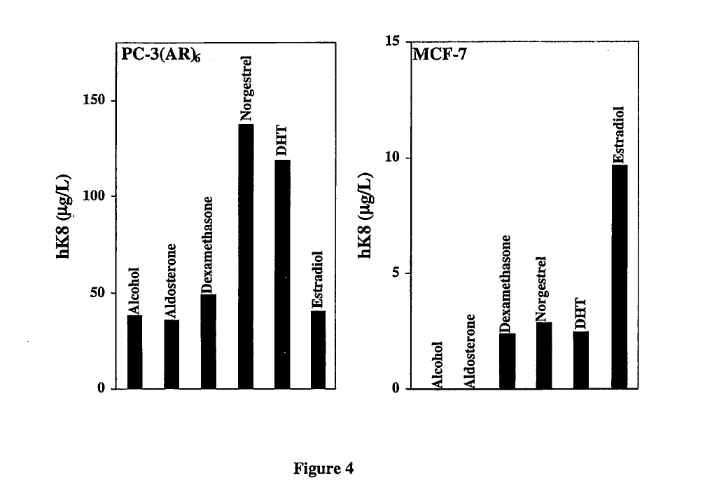

Production of hK8 by cancer cell lines and hormonal regulation Human kallikrein 8 is predicted to be a secreted protein. Breast, ovarian and prostate cancer cell lines were cultured, stimulated with various steroids at 10⁻⁸ mol/L final concentration and the tissue culture supernatants were analyzed after 7 days incubation with the developed assay. Among all cell lines, MDA-MB-231, BT-474, LNCaP and ZR-75 did not produce detectable amounts of hK8, either before or after stimulation with the five different steroids, at 10⁻⁸ M. Detectable hK8 was found in supernatants from the cell lines PC-3 (AR)₆ [range 38-156 μg/L], BG-1 [range 48-65 μg/L], Caov-3 [range 9-15 μg/L], MCF-7 [range 1-10 μg/L] and T-47D [range 1-5 μg/L]. In terms of hormonal stimulation, the steroid(s) that produced a significant increase (at least 2-fold) of baseline hK8 concentration (alcohol stimulation) were norgestrel and dihydrotestosterone for PC-3 (AR)ᵣ cells (3.5-fold increase over control), estradiol for T-47D cells (3.5-fold increase over control) and dexamethasone (4-fold), norgestrel (4-fold), dihydrotestosterone (3-fold) and estradiol (10-fold) for MCF-7 cells. For the cell lines BG-1 and Caov-3, there was not much change in hK8 concentration with any of the tested steroids. These data suggest that KLK8 gene expression can be significantly up-regulated by a variety of steroids, including androgens, glucocorticoids and estrogens in different cancer cell lines. Examples of steroid hormone regulation of the KLK8 gene in the cell lines PC-3 (AR)₆ and MCF-7 are shown in FIG. 4.

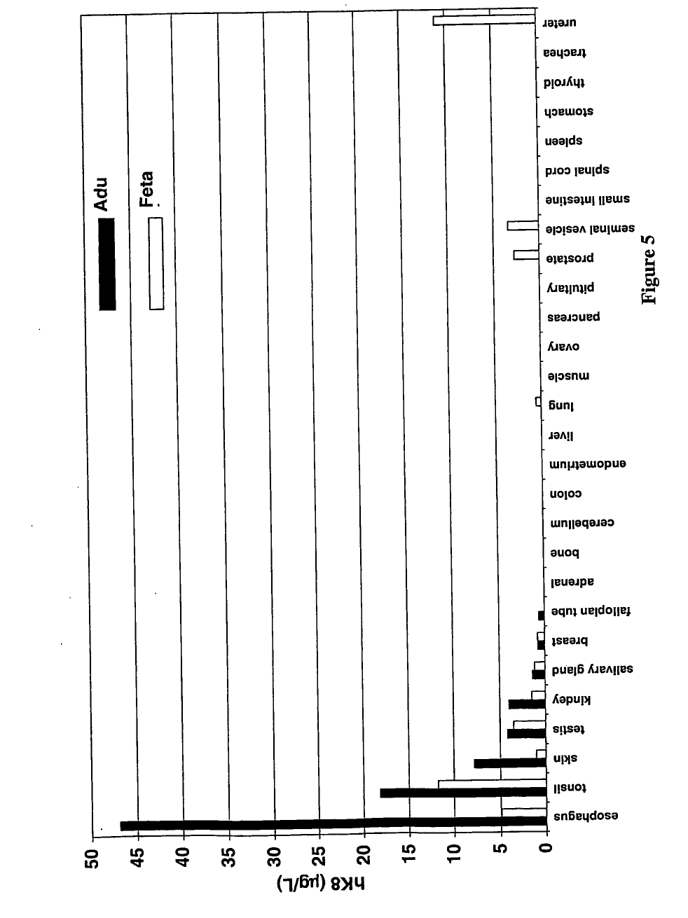

hK8 in human tissue, extracts. hK8 was quantified in various adult and fetal male and female tissue extracts (Table 2). The data are presented graphically in FIG. 5. The highest levels were seen in esophagus, followed by tonsil, skin, testis, kidney, salivary gland, breast and fallopian tube. In general, the positivity for hK8 was seen in both the adult and fetal tissues, with the exception of fetal lung, prostate, seminal vesicle and ureter. These tissues seem to express significantly more hK8 during the fetal period.

Ascites fluid of ovarian cancer patients. hK8 was analyzed in 31 ascites fluid samples obtained from patients with metastatic ovarian carcinoma. All samples were positive for hK8. The following statistical parameters describe the findings. Lowest value, 5 μg/L; Highest value, 487 μg/L; Mean±standard, deviation, 129±149 μg/L; Median 62 μg/L.

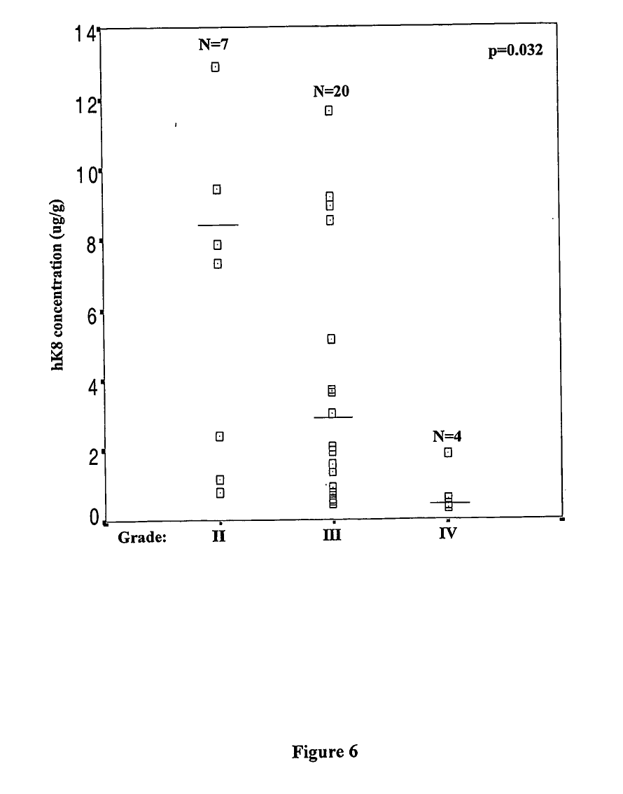

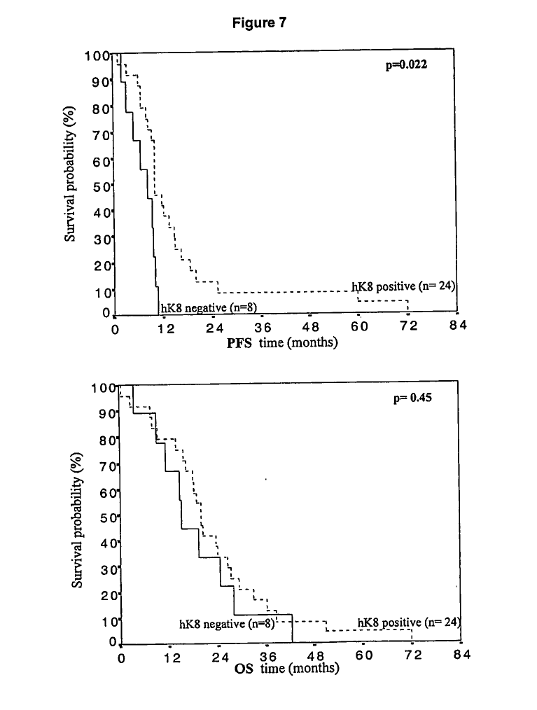

After correcting for total protein in these ascites samples, the hK8 concentration, expressed as μg of hK8 per g of total protein was 3.51±0.66 μg/g (mean±SD), the range was 0.34-12.9 μg/g and the median was 1.73 μg/g. For these patients, information on age, serum CA125, various clinicopathological variables and outcomes (progression-free and overall survival) was also available. All patients had stage III/IV disease. There was no association found between ascites fluid hK8 levels and either patient age or serum CA125 (data not shown). However, there was an inverse association between ascites fluid hK8 and tumor grade (FIG. 6) as well as progression-free survival (PFS), but not overall survival (OS) (FIG. 7).

Ovarian cancer cytosolic extracts. Extracts from ovarian cancer tissues were analyzed for hK8. Among twenty extracts, one was negative and 19 were positive, with values ranging from 0.3 to 500 μg/L. Three normal ovarian tissue extracts had a concentration of 0-0.16 μg/L. All extracts were adjusted to the same total protein concentration (1 mg/mL). Thus, hK8 appears to be highly overexpressed in more than 90% of ovarian cancer tissues. The mean±standard deviation for hK8 concentration in ovarian cancer tissue extracts was 64±77 μg/L and the median was 34 μg/L.

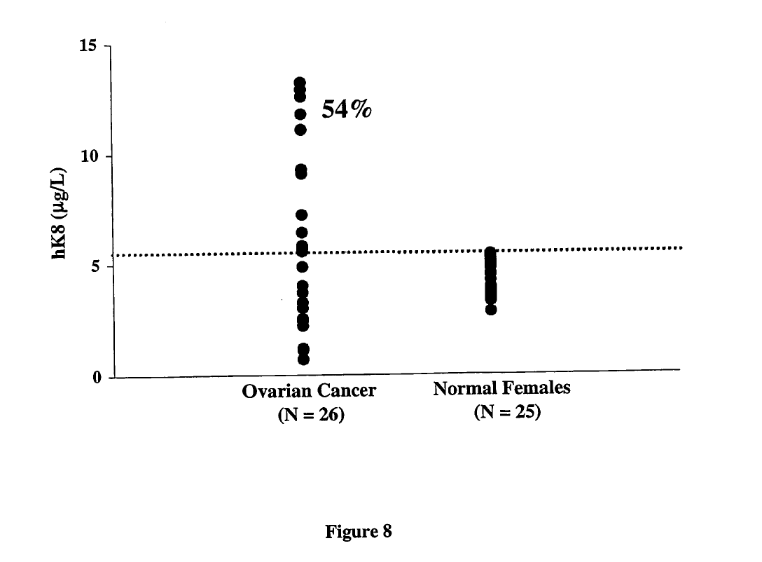

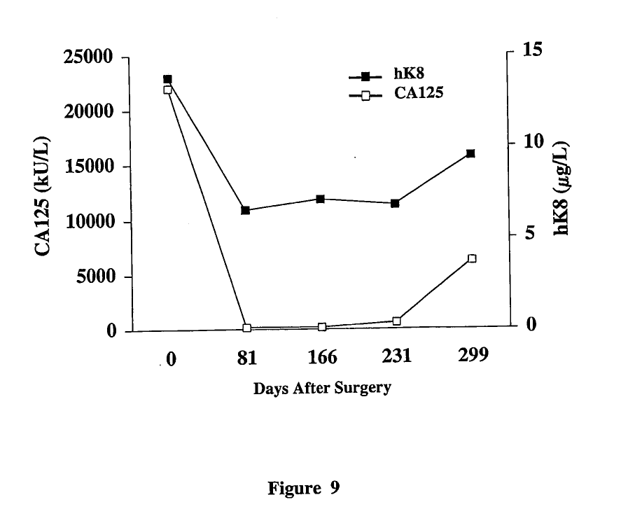

Serum of cancer patients. A total of 36 serum samples from patients with various malignancies, including prostate (n=6), breast (n=6), liver (n=6), testicular (n=6), colon (n=6) and ovarian cancer (n=6) along with 6 serum samples from healthy male and 10 serum samples from healthy female subjects were analyzed. The highest level in normals was 5 μg/L. Among patients with cancer, one patient with breast cancer (8.2 μg/L) and one patient with colon cancer (7.4 μg/L) had elevated hK8 levels. However, in the ovarian cancer group, 4 patients had elevated levels (6.4-12.9 μg/L). In view of this finding, another 16 sera from normal females and another 20 sera from pre-surgical ovarian cancer patients of various stages were analyzed. The combined data from 26 control women and 26 women with ovarian cancer are shown in FIG. 8. When a cutoff of 5.5 μg/L is used for classification, (95% specificity), the positivity rate (sensitivity) of this test for ovarian cancer patients is approximately 54% (FIG. 8). A series of serum samples obtained from one patient over approximately 1 year (FIG. 9) were further analyzed. After surgery, CA125 and hK8 levels dropped and then started to increase again, approximately 231 days post-surgery.

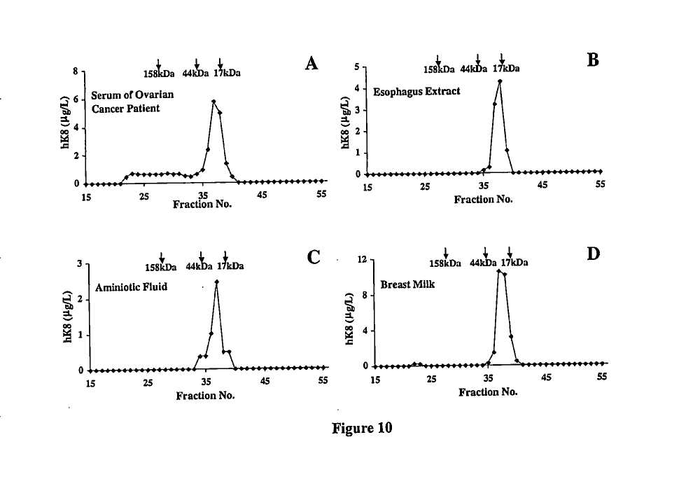

High-performance liquid chromatography. Many enzymes circulate in serum as complexes with proteinase inhibitors. For some kallikreins, including prostate-specific antigen, the major circulating form is a complex with a proteinase inhibitor (e.g. PSA is complexed with alpha-1 antichymotrypsin). In order to investigate if hK8 in biological fluids and tissue extracts is circulating in various molecular forms, one amniotic fluid sample, one milk, an esophageal extract and a serum of an ovarian cancer patient with elevated hK8, were fractionated with gel filtration chromatography, as described earlier (21) and all fractions were analyzed with the hK8 immunoassay. The results are shown in FIG. 10. In all cases, the immunoreactivity elutes as a single peak with a molecular weight of approximately 30 kDa, consistent with the molecular weight of free (unbound) hK8. Thus, hK8 circulates in biological fluids, including serum in its free, uncomplex form.

Discussion

Generally, extracellular serine proteases are translated as pre-proproteins and secreted as proproteins in the extracellular space. hK8 is a predicted extracellular protease because its amino terminal end contains a hydrophobic sequence resembling a signal peptide, similar to all other members of this family (9;22). hK8 (human neuropsin) has 72% identity to mouse neuropsin at the amino acid level. Recombinant mouse neuropsin was produced using a baculovirus expression system and was found to be secreted into the culture medium as a proprotein, containing the tetrapeptide (Glu-Gly-Ser-Lys) at the amino terminus (23). The homologous amino terminus in human neuropsin would contain the tetrapeptide (Glu-Gln-Asp-Lys). If this propeptide is removed by another protease, hK8 is activated (23). The crystal structure of mouse neuropsin has now been reported (24). hK8 (human neuropsin) and mouse neuropsin share a potential N-glycosylation site (Asn-X-Ser) at the same position on their amino acid sequences. As mouse neuropsin produced by the baculovirus expression system has N-glycans at this site (25;26), the purified human hK8 is expected to also be glycosylated at this site.

The studies described herein confirm that the developed immunoassay is highly specific for hK8. The detection of hK8 in many biological fluids further establishes experimentally that hK8 is an extracellular serine protease in vivo. hK8 concentration in breast milk is much higher than 100-fold higher than levels of the homologous kallikreins, hK10 and hK11 (6;18;18). Since other kallikreins have been found to be produced and secreted by epithelial cells (27), it is conceivable that hK8 is produced by breast epithelial cells in response to hormonal stimuli (1;2). The expression of hK8 in brain and other tissues was reported previously at the mRNA level (1;2;17). Although hK8 transcripts are expressed in pancreas, brain, and placenta, the expression of hK8 protein seen in esophagus, tonsil, skin, testis, kidney, salivary gland, breast and fallopian tube extracts have not been reported before.

The low recoveries in male and female sera may be attributable to sequestration of hK8 by protease inhibitors, similar to the situation with other kallikreins (4-6).

It is known that at least two human kallikreins, hK2 and hK3 (PSA) form complexes in serum and tissues with protease inhibitors (28-31). These complexes contribute to the lower than expected recoveries of these analytes in serum (32). Here, recovery of added recombinant hK8 to male and female serum is around 40-70%, close to the recoveries of hK3 (PSA) in serum (32). These data support the proposal for hK8 binding to proteinase inhibitors. In breast cytosols and seminal plasma, recovery was close to 100%.

In conclusion, this is the first report of the production and purification of human kallikrein 8 in a baculovirus system. The recombinant hK8 has been used to develop a highly sensitive and specific immunoassay which, in turn, was utilized to demonstrate presence of hK8 in biological fluids and tissue extracts.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

Below full citations are set out for the references referred to in the specification.

TABLE 1
Purification of recombinant hK8 produced
by the baculovirus system

| | Total | | | |
|---|---|---|---|---|
| Purification | protein | hK8 | Recovery | Purification |
| step | (μg)^{a} | (μg)^{b} | (%) | (fold) |
| Culture medium | 359 × 10³ | 1411^{c} | 100 | 1 |
| SP Sepharose | 600 | 334 | 23.7 | 142 |
| Heparin | 163 | 154 | 10.9 | 240 |
| Benzamidine | 108 | 108 | 7.7 | 254 |

^{a}Evaluated by the bicinchoninic acid (BCA) kit with albumin as standard (Pierce Chemical Co., Rockford, IL).
^{b}Evaluated by Western blot band intensities on an X-ray film. See text for details.
^{c}Based on a 400 mL culture medium.

TABLE 2
hK8 concentration in biological fluids

| | Samples | hK8 Concentration (μg/L) | | | | Positivity |
|---|---|---|---|---|---|---|
| Fluid | tested | Range | Mean (SD) | | Median | rate (%) |
| Amniotic fluid | 10 | 1.0-22 | 6.7 | (6.5) | 5.6 | 100 |
| Breast milk | 13 | 17-2665 | 599 | (840) | 174 | 100 |
| Cerebrospinal fluid | 13 | 0.0^{a}-1.4 | 0.4 | (0.4) | 0.3 | 69 |
| Follicular fluid | 5 | 1.4-5.4 | 3.1 | (1.5) | 3.0 | 100 |
| Seminal plasma | 13 | 1.0-7.4 | 4.3 | (3.6) | 2.8 | 100 |
| Serum (male) | 10 | 2.0-6.9 | 3.9 | (1.5) | 3.6 | 100 |
| (female) | 25 | 0.4-6.0 | 2.2 | (2.3) | 2.3 | 100 |

^{a}Concentrations < 0.2 μg/L are shown as zero.

### FULL CITATIONS FOR REFERENCES REFERRED TO IN THE SPECIFICATION

1. Diamandis E P, Yousef G M, Luo L Y, Magklara A, Obiezu C V. The new human kallikrein gene family: implications in carcinogenesis. Trends Endocrinol Metab 2000; 11:54-60.
2. Yousef G M, Diamandis E P. The new human tissue kallikrein gene family: structure, function, and association to disease. Endocr Rev 2001; 22:184-204.
3. Stamey T A, Yang N, Hay A R, McNeal J E, Freiha F S, Redwine E. Prostate-specific antigen as a serum marker for adenocarcinoma of the prostate. N Engl J Med 1987; 317:909-16.
4. Diamandis E P, Yousef G M, Soosaipillai A R, Bunting P. Human kallikrein 6 (zyme/protease M/neurosin): a new serum biomarker of ovarian carcinoma. Clin Biochem 2000; 33:579-83.
5. Luo L Y, Bunting P, Scorilas A, Diamandis E P. Human kallikrein 10: a novel tumor marker for ovarian carcinoma? Clin Chim Acta 2001; 306:111-8.
6. Diamandis E P, Okui A, Mitsui S, Luo L Y, Soosaipillai A, Grass L et al. Human kallikrein 11: a new biomarker of prostate and ovarian carcinoma. Cancer Res 2002; 62:295-300.
7. Diamandis E P, Yousef G M. Human tissue kallikrein gene family: a rich source of novel disease biomarkers. Expert Rev Mol Diagn 2001; 1:182-90.
8. Diamandis E P, Yousef G M, Clements J, Ashworth L K, Yoshida S, Egelrud T et al. New nomenclature for the human tissue kallikrein gene family. Clin Chem 2000; 46:1855-8.
9. Yoshida S, Taniguchi M, Hirata A, Shiosaka S. Sequence analysis and expression of human neuropsin cDNA and gene. Gene 1998; 213:9-16.
10. Chen Z L, Yoshida S, Kato K, Momota Y, Suzuki J, Tanaka T et al. Expression and activity-dependent changes of a novel limbic-serine protease gene in the hippocampus. J Neurosci 1995; 15:5088-97.
11. Okabe A, Momota Y, Yoshida S, Hirata A, Ito J, Nishino H, Shiosaka S. Kindling induces neuropsin mRNA in the mouse brain. Brain Res 1996; 728:116-20.
12. Momota Y, Yoshida S, Ito J, Shibata M, Kato K, Sakurai K et al. Blockade of neuropsin, a serine protease, ameliorates kindling epilepsy. Eur J Neurosci 1998; 10:760-4.
13. Komai S, Matsuyama T, Matsumoto K, Kato K, Kobayashi M, Imamura K et al. Neuropsin regulates an early phase of schaffer-collateral long-term potentiation in the murine hippocampus. Eur J Neurosci 2000; 12:1479-86.
14. Shimizu-Okabe C, Yousef G M, Diamandis E P, Yoshida S, Shiosaka S, Fahnestock M. Expression of the kallikrein gene family in normal and Alzheimer's disease brain. Neuroreport 2001; 12:2747-51.
15. Underwood L J, Tanimoto H, Wang Y, Shigemasa K, Parmley T H, O'Brien T J. Cloning of tumor-associated differentially expressed gene-14, a novel serine protease overexpressed by ovarian carcinoma Cancer Res 1999; 59:4435-9.
16. Magklara A, Scorilas A, Katsaros D, Massobrio M, Yousef G M, Fracchioli S et al. The human KLK8 (neuropsin/ovasin) gene: identification of two novel splice variants and its prognostic value in ovarian cancer. Clin Cancer Res 2001; 7:806-11.
17. Mitsui S, Tsuruoka N, Yamashiro K, Nakazato H, Yamaguchi N. A novel form of human neuropsin, a brain-related serine protease, is generated by alternative splicing and is expressed preferentially in human adult brain. Eur J Biochem 1999; 260:627-34.
18. Luo L Y, Grass L, Howarth D J, Thibault P, Ong H, Diamandis E P. Immunofluorometric assay of human kallikrein 10 and its identification in biological fluids and tissues. Clin Chem 2001; 47:237-46.
19. Christopoulos T K, Diamandis E P. Enzymatically amplified time-resolved fluorescence immunoassay with terbium chelates. Anal Chem 1992; 64:342-6.
20. Hassapoglidou S, Diamandis E P, Sutherland D J. Quantification of p53 protein in tumor cell lines, breast tissue extracts and serum with time-resolved immunofluorometry. Oncogene 1993; 8:1501-9.
21. Yu H, Diamandis E P. Ultrasensitive time-resolved immunofluorometric assay of prostate-specific antigen in serum and preliminary clinical studies. Clin Chem 1993; 39:2108-14.
22. Yousef G M, Diamandis E P. The new kallikrein-like gene, KLK-L2. Molecular characterization, mapping, tissue expression, and hormonal regulation. J Biol Chem 1999; 274:37511-6.
23. Shimizu C, Yoshida S, Shibata M, Kato K, Momota Y, Matsumoto K et al. Characterization of recombinant and brain neuropsin, a plasticity-related serine protease. J Biol Chem 1998; 273:11189-96.
24. Kishi T, Kato M, Shimizu T, Kato K, Matsumoto K, Yoshida S et al. Crystallization and preliminary X-ray analysis of neuropsin, a serine protease expressed in the limbic system of mouse brain. J StructBiol 1997; 118:248-51.
25. Kishi T, Kato M, Shimizu T, Kato K, Matsumoto K, Yoshida S et al. Crystal structure of neuropsin, a hippocampal protease involved in kindling epileptogenesis. J Biol Chem 1999; 274:4220-4.
26. Takahashi N, Tsukamoto Y, Shiosaka S, Kishi T, Hakoshima T, Arata Y et al. N-glycan structures of murine hippocampus serine protease, neuropsin, produced in Trichoplusia ni cells. Glycoconj J 1999; 16:405-14.
27. Petraki C D, Karavana V N, Skoufogiannis P T, Little S P, Howarth D J, Yousef G M, Diamandis E P. The spectrum of human kallikrein 6 (zyme/protease M/neurosin) expression in human tissues as assessed by immunohistochemistry. J Histochem Cytochem 2001; 49:1431-42.
28. Christensson A, Laurell C B, Lilja H. Enzymatic activity of prostate-specific antigen and its reactions with extracellular serine proteinase inhibitors. Eur J Biochem 1990; 194:755-63.
29. Stenman U H, Leinonen J, Alfthan H, Rannikko S, Tuhkanen K, Alfthan O. A complex between prostate-specific antigen and alpha 1-antichymotrypsin is the major form of prostate-specific antigen in serum of patients with prostatic cancer: assay of the complex improves clinical sensitivity for cancer. Cancer Res 1991; 51:222-26.
30. Stephan C, Jung K, Lein M, Sinha P, Schnorr D, Loening S A. Molecular forms of prostate-specific antigen and human kallikrein 2 as promising tools for early diagnosis of prostate cancer. Cancer Epidemiol Biomarkers Prev 2000; 9:1133-47.
31. Saedi M S, Zhu Z, Marker K, Liu R S, Carpenter P M, Rittenhouse H, Mikolajczyk S D. Human kallikrein 2 (hK2), but not prostate-specific antigen (PSA), rapidly complexes with protease inhibitor 6 (PI-6) released from prostate carcinoma ceus. Int J Cancer 2001; 94:558-63.
32. Ferguson R A, Yu H, Kalyvas M, Zammit S, Diamandis E P. Ultrasensitive detection of prostate specific antigen by a time-resolved immunofluorometric assay and the Immulite® Immunochemiluminescent third generation assay: potential applications in prostate and breast cancers. Clin Chem 1996; 42:675-684.

1. A method for detecting a kallikrein 8 polypeptide associated with ovarian cancer in a patient comprising:
(a) obtaining a sample from a patient;
(b) detecting in the sample kallikrein 8 polypeptide; and
(c) comparing the detected amounts with amounts detected for a standard.

2. A method for diagnosing and monitoring ovarian cancer in a subject comprising detecting in a sample from the subject a kallikrein 8 polypeptide.

3. A method of detecting ovarian cancer in a patient, the method comprising comparing:
(a) levels of a kallikrein 8 polypeptide in a sample from the patient; and
(b) normal levels of expression of kallikrein 8 polypeptide in a control sample, wherein a signficant difference in the levels of kallikrein 8 polypeptides, relative to the corresponding normal levels, is indicative of ovarian cancer.

4. A method for monitoring the progression of ovarian cancer in a patient, the method comprising: (a) detecting in a sample from the patient at a first time point, a kallikrein 8 polypeptide; (b) repeating step (a) at a subsequent point in time; and (c) comparing levels detected in steps (a) and (b), and thereby monitoring the progression of ovarian cancer.

5. A method for determining in a patient whether ovarian cancer has metastasized or is likely to metastasize in the future, the method comprising comparing (a) levels of a kallikrein 8 polypeptide in a patient sample; and (b) normal levels or non-metastatic levels of a kallikrein 8 polypeptide, in a control sample wherein a significant difference between levels of expression in the patient sample and the normal levels or non-metastatic levels is an indication that the ovarian cancer has metastasized.

6. A method for assessing the aggressiveness of indolence of ovarian cancer comprising comparing: (a) levels of expression of a kallikrein 8 polypeptide in a patient sample and (b) normal levels of expression of the kallikrein 8 polypeptide, in a control sample, wherein a significant difference between the levels in the patient sample and normal levels is an idication that the cancer is aggressive or indolent.

7. A method for assessing the potential efficacy of a test agent for inhibiting ovarian cancer in a patient, the method comprising comparing: (a) levels of a kallikrein 8 polypeptide in a first sample obtained from a patient and exposed to the test agent, and (b) levels of the kallikrein 8 polypeptide in a second sample obtained from the patient, wherein the sample is not exposed to the test agent, wherein a significant difference in the levels of expression of the kallikrein 8 polypeptide in the first sample, relative to the second sample, is an indication that the test agent is potentially efficacious for inhibiting ovarian cancer in the patient.

8. A method of claim 7 wherein the first and second samples are portions of a single sample obtained from the patient.

9. A method of claim 7 wherein the first and second samples are portions of pooled samples obtained from the patient.

10. A method of assessing the efficacy of a therapy for inhibiting ovarian cancer in a patient, the method comprising comparing; (a) levels of a kallikrein 8 polypeptide in a first sample obtained from the patient, and (b) levels of the kallikrein 8 polypeptide in a second sample obtained from the patient following therapy, wherein a significant difference in the levels of expression of the kallikrein 8 polypeptide in the second sample relative to the first sample, in an indication that the therapy is efficacious for inhibiting ovarian cancer in the patient.

11. A method of selecting an agent for inhibiting ovarian cancer in a patient the method comprising (a) obtaining a sample comprising cancer cells from the patient; (b) separately exposing aliquots of the sample in the presence of a plurality of test agents; (c) comparing levels of kallikrein 8 polypeptide in each of the aliquots; and (d) selecting one of the test agents which alters the levels of kallikrein 8 polypeptide in the aliquot containing that test agent, relative to other test agents.

12. A method of inhibiting ovarian cancer in a patient, the method comprising (a) obtaining a sample comprising cancer cells from the patient; (b) separately maintaining aliquots of the sample in the presence of a plurality of test agents; (c) comparing levels of kallikrein 8 polypeptide in each of the aliquots, and (d) administering to the patient at least one of the test agents which alters the levels of kallikrein 8 polypeptide in the aliquot containg that test agent, relative to other test agents.

13. A method of assessing the ovarian cancer cell carcinogenic potential of a test compound, the method comprising: (a) maintaining separate aliquots of ovarian cancer cells in the presence and absence of the test compound; and (b) comparing expression of a kallikrein 8 polypeptide in each of the aliquots, and wherein a significant difference in levels of kallikrein 8 polypeptide in the aliquot maintained in the presence of the test compound, relative to the aliquot maintained in the absence of the test compound, is an indication that the test compound possesses ovarian cancer cell cacinogenic potential.

14. A method of claim 1 wherein the patient sample comprises serum obtained from the patient.

15. A method of claim 1 wherein the kallikrein 8 polypeptide is detected using antibodies that bind to a kallikrein 8 polypeptide or part thereof.

16. A method of claim 15 wherein the anitbodies are used in an immunoassay.

17. A method as claimed in claim 1 which further comprises detecting one or more of human stratum corneum chymotryptic enzyme (HSCCE), kallikrein 2, kallikrein 4, kallikrein 5, kallikrein 6, kallikrein 9, kallikrein 10, kallikrein 11, CA125, CA15-3, CA72-4, CA19-9, OVX1, lysophosphatidic acid (LPA), creatin-kinase BB, haptoglobin alpha, prostasin, osteopontin, and carcinoembryonic antigen (CEA).

18. A method for screening a subject for ovarian cancer comprising:
(a) incubating a biological sample from the subject with a first antibody specific for hK8 which is directly or indirectly labeled with a detectable substance, and a second antibody specific for hK8 which is immobilized;
(b) separating the first antibody from the second antibody to provide a first antibody phase and a second antibody phase;
(c) detecting the detectable substance in the first or second antibody phase thereby quantitating hK8 in the biological sample; and
(d) comparing the quantitated hK8 with levels for a predetermined standard.

19. An in vivo method for imaging ovarian cancer comprising:
(a) injecting a patient with an agent that binds to a kallikrein 8 polypeptide, the agent carryig a label for imaging the ovarian cancer;
(b) allowing the agent to incubate in vivo and bind to a kallikrein 8 polypeptide associated with the ovarian cancer; and
(c) detecting the presence of the label localized to the ovarian cancer.

20. A method as claimed in claim 19 wherein the agent is an antibody which recognizes a kallikrein 8 polypeptide.

21. A method as claimed in claim 19 wherein the label is a radiolabel, fluorescent label, nuclear magnetic resonance active label, positron emitting isotope detectable by a positron emission tomography (“PET”) scanner, chemiluminescer, or enzymatic marker.

22. A kit for carrying out a method as claimed in claim 19.

23. A kit for assessing whether a patient is afflicted with ovarian cancer, the kit comprising reagents that specifically bind with kallikrein 8 polypeptides.

24. A kit for assessing the suitability of each of a plurality of agents for inhibiting ovarian cancer in a patient, the kit comprising: (a) the plurality of agents; and (b) reagents for detecting a kallikrein 8 polypeptide.

25. A kit as claimed in claim 23 wherein the reagents are antibodies that specifically bind with protein or protein fragments corresponding to a kallikrein 8 polypeptide.

26. A medium for holding instructions for performing a method for determining whether a patient has ovarian cancer or a pre-disposition to ovarian cancer, comprising determining the presence or absence of a kallikrein 8 polypeptide, and based on the presence or absence of a kallikrein 8 polypeptide, determining whether the patient has ovarian cancer or a pre-disposition to ovarian cancer, and optionally recommending treatment for the ovarian cancer or pre-ovarian cancer condition.

27. A method in an electronic system and/or in a network for determing whether a subject has ovarian cancer or a pre-disposition to ovarian cancer associated with a kallikrein 8 polypeptide comprising determining the presence or absence of a kallikrein 8 polypeptide, and based on the presence or absence of the kallikrein 8 polypeptide, determing whether the subject has ovarian cancer or a pre-disposition to ovarian cancer, and optionally recommending treatment for the ovarian cancer or pre-ovarian cancer condition.

28. A method of treating a patient susceptible to, or having a cancer that expresses a kallikrein 8 polypeptide comprising administering to the patient an effective amount of antibodies that bind specifically to a kallikrein 8 polypeptide.

29. A method of inhibiting the growth of tumor cells expressing a kallikrein 8 polypeptide, comprising administering to a patient antibodies which bind specifically to a kallikrein 8 polypeptide in amounts effective to inhibit growth of the tumor cells.


SEQUENCE LISTING (4 sequences)

SEQ ID NO: 1 - protein, 164 aa, Homo sapiens
MGRPRPRAAKTWMFLLLLGGAWAGHSRAQEDKVLGGHECQPHSQPWQAALFQGQQLLCGGVLVGGNWVLTAAHCKKPKYTVRLGDHSLQNKDGPEQEIPVVQSIPHPCYNSSDVEDHNHDLMLLQLRDQASLGSKVKPISLADHCTQPGQKCTVSGWGTVTSPR

SEQ ID NO: 2 - protein, 260 aa, Homo sapiens
MGRPRPRAAKTWMFLLLLGGAWAGHSRAQEDKVLGGHECQPHSQPWQAALFQGQQLLCGGVLVGGNWVLTAAHCKKPKYTVRLGDHSLQNKDGPEQEIPVVQSIPHPCYNSSDVEDHNHDLMLLQLRDQASLGSKVKPISLADHCTQPGQKCTVSGWGTVTSPRENFPDTLNCAEVKIFPQKKCEDAYPGQITDGMVCAGSSKGADTCQGDSGGPLVCDGALQGITSWGSDPCGRSDKPGVYTNICRYLDWIKKIIGSKG

SEQ ID NO: 3 - DNA, 493 bp, Homo sapiens
atgggacgcccccgacctcgtgcggccaagacgtggatgttcctgctcttgctggggggagcctgggcaggacactccagggcacaggaggacaaggtgctggggggtcatgagtgccaaccccattcgcagccttggcaggcggccttgttccagggccagcaactactctgtggcggtgtccttgtaggtggcaactgggtccttacagctgcccactgtaaaaaaccgaaatacacagtacgcctgggagaccacagcctacagaataaagatggcccagagcaagaaatacctgtggttcagtccatcccacacccctgctacaacagcagcgatgtggaggaccacaaccatgatctgatgcttcttcaattgcgtgaccaggcatccctggggtccaaagtgaagcccatcagcctggcagatcattgcacccagcctggccagaagtgcaccgtctcaggctggggcactgtcaccagtccccgag

SEQ ID NO: 4 - DNA, 783 bp, Homo sapiens
atgggacgcccccgacctcgtgcggccaagacgtggatgttcctgctcttgctggggggagcctgggcaggacactccagggcacaggaggacaaggtgctggggggtcatgagtgccaaccccattcgcagccttggcaggcggccttgttccagggccagcaactactctgtggcggtgtccttgtaggtggcaactgggtccttacagctgcccactgtaaaaaaccgaaatacacagtacgcctgggagaccacagcctacagaataaagatggcccagagcaagaaatacctgtggttcagtccatcccacacccctgctacaacagcagcgatgtggaggaccacaaccatgatctgatgcttcttcaactgcgtgaccaggcatccctggggtccaaagtgaagcccatcagcctggcagatcattgcacccagcctggccagaagtgcaccgtctcaggctggggcactgtcaccagtccccgagagaattttcctgacactctcaactgtgcagaagtaaaaatctttccccagaagaagtgtgaggatgcttacccggggcagatcacagatggcatggtctgtgcaggcagcagcaaaggggctgacacgtgccagggcgattctggaggccccctggtgtgtgatggtgcactccagggcatcacatcctggggctcagacccctgtgggaggtccgacaaacctggcgtctataccaacatctgccgctacctggactggatcaagaagatcataggcagcaagggctga